(19) United States
(12) Patent Application Publication
(10) Pub. No.: US 2011/0207993 A1
(43) Pub. Date: Aug. 25, 2011

(54) METHOD FOR ARTIFICIAL INSEMINATION OF COWS

(76) Inventor: Carlos Alberto BARCELO ROJAS, Villahermosa (MX)

(21) Appl. No.: 12/884,039

(22) Filed: Sep. 16, 2010

Related U.S. Application Data
(60) Provisional application No. 61/242,991, filed on Sep. 16, 2009.

(51) Int. Cl. A61B 17/43

(52) U.S. Cl. 600/35

(57) ABSTRACT

The present invention is related to a method of artificial animal reproduction by which healthy cows and heifers are impregnated on the first attempt at artificial insemination. The sperm is deposited at the time the uterine horns are at the optimum time to conceive.

### REFERENCE RELATED APPLICATIONS

This application is a non-provisional application based on the provisional application of EU No. 61/242,991 filed on Sep. 16, 2009, the content of which is incorporated by reference in this application.

### FIELD OF THE INVENTION

The method of the present invention is related to a method of artificial insemination to impregnate any healthy cow or heifer in the first attempt.

### BACKGROUND OF THE INVENTION

The conventional recto-vaginal method of artificial insemination has shown higher rates of conception in cattle when compared with the vaginal method, and the use of the vaginoscopy. This can be seen in multiple studies developed around the world as published in the Handbook of artificial insemination of the “Universidad Nacional de Tucuman” in the Republic of Argentina in we can see the percentage of non-return obtained with each method:

| Method | N° of breeding stock | % of non-return in 60 days |
|---|---|---|
| Recto-vaginal | 751 ¹ | 55.00 |
| Vaginal | 757 ¹ | 50.02 |
| Recto-vaginal | 319 ² | 53.3 |
| Vaginal | 319 ² | 37.2 |

¹ = Cows
² = Heifers

The recto-vaginal technique is of universal use and consists of introducing an applicator through the vagina containing the semen, guided into the cervix by means of a gloved hand through the rectum to hold the cervix, and to guide the fingers of the gloved hand through the cervical canal until the tip of the applicator reaches the body of the uterus where the semen is deposited.

This model suggests two critical points that define success: 1°.—The heat detection; y 2°.—Good timing for insemination.

As for the heat detection is concerned and regardless of the system used to detect, its importance lies in establishing the best possible moment of insemination based on the time that is observed that the heat starts. Basically, the traditional known method indicates that cows showing estrus in the morning should be inseminated in the late evening of the same day. Cows showing estrus in the afternoon should be inseminated early morning the next day.

Unfortunately, most of the studies carried out in the field of determining the optimal moment for insemination, point out that both early insemination (that is 6 hours or less after heat) as well as late insemination (6 hours or more after heat) results in low conception rate.

In early insemination most of the sperm reach the egg alive, but exhausted, without force and little chance of a successful fertilization, in late insemination the sperms are limited by the delay of the upload mechanisms. These two factors point that the vast majority of the researchers indicate that the best moment to inseminate the animal is close to the end of the heat. (Handbook of artificial insemination, Universidad Nacional de Tucumán).

Table 1 shows a diagram of the relationship between the moment of the artificial insemination and fertility rate, cited in the Handbook of artificial insemination Universidad Nacional de Tucumán. As observed in the diagram it is possible to obtain maximum conception through artificial insemination. However evidence from the reports with the recto-vaginal method demonstrates that this is not possible and that in any way it is possible to match what can be obtained using fertile bulls. That is why the use of artificial insemination is declining. (Hoard's Dairyman magazine, March 1999), though it is true that on the one hand makes the genetic advance possible, on the other hand it produces devastation in the production of calves or of milk. As long as we continue to use the traditional method of inseminating in a conveniently fixed but ineffective schedule, many cows will not impregnate on the first attempt. It will fail not because of the quality of the semen, the skill of the inseminator, stress, or because the cow's clitoris has not been rubbed, but for improper timing.

Another consideration related to detection of heat is managing the herd to implement this operation—beef, dairy cows or both. Generally speaking, animals in the herd are divided in two groups, impregnated cows and non-pregnant cows. If the non-pregnant cows are left grazing, in general they have to be observed in the morning and in the evening in a specific area of the lot to detect the heat. This represents, in humid zones, to expose the cows to quagmires and the consistent difficulty to handle the cattle. In the arid or semiarid zones, this represents the creation of special areas to detect heats and the consistent need to supply food. On the other hand, if the non-pregnant cows are fed and handled in confinement since is the habit in the majority of the dairy specialized herds, this operation does not represent big problems since in general it is done in the same corrals where it feeds to the cattle.

### SUMMARY OF THE INVENTION

This invention refers to an artificial animal reproduction procedure whereby healthy heifers or cows are impregnated at the first attempt of insemination because the semen is deposited at the time it gets to the uterine horns, the optimum time to deposit the sperm in the oviduct.

This method is based on:

A).—The Diagnosis of the Horn where the Ovulation Will Occur.

This process of diagnosis starts when discovering the horn that has the ovary with the active follicle is of major diameter. This is a temporary condition only evident after the heat or sexual receptivity has finished, just before ovulation. It is based on an exploration to hold, align and stimulate the uterus, in strategic points, to properly analyze the diameter between the horns to estimate the difference. The first point corresponds to a test that is performed at the base of the horns, known as the body of the uterus. This test should stimulate the uterus by rubbing to cause an erection that allows us to see and compare the diameter between the horns at the height of the body of the uterus. The uterus must pose horizontally aligned on the middle, annular and index fingers while the thumb runs, analyzes and compares the diameter between the horns; the second point of comparative analysis in diameter between the horns, is located just in front of where starts the external fork of the horns. This is carried out after stimulating the horns rubbing them to cause an erection, taking special care to make sure you stretch and horizontally align the horns to make comparative analysis of diameter. Finally, and only when, a difference cannot be established, the comparative analysis must be at the height of the midpoint of the largest curve of the horns, taking special care of causing an erection, and stretch and horizontally align the curvature on the fingers index, middle and ring, in order to be sure that both horns are being compared in the right place.

B).—Diagnosis on the Horns of the Range and the Optimal Moment for Insemination.

Refers to the way of predicting the phase to find the active follicle through the behavior of the horns to a predetermined manual stimulus, because all the changes of the follicle are associated with hormonal processes that are manifested in the uterine behavior in one of two ways: The first with a violent erection that stands out because it conforms as soon as we touch into the uterus to try to locate it. The characteristic feature of this stage is that the local stimulation in any part of the uterus has the ability to extend the erection to the rest of the organ, and the ability to maintain its intensity by the mere fact of holding it on the hand. It indicates that the follicle is at the maturity stage, is a uterus that at rest is flaccid but clearly shows its tubular configuration coiled on itself. It is a uterus which transforms its aspect to the touch, from flabby to tense, showing its status of excitement with a violent erection, both horns form a perfect tubular form of conical aspect to the tip, and the tense of the contour, does not allow the friction between the walls the first time that it is rubbed. At this stage, you can be sure that the follicle is still maturing, and that you can calmly wait six hours more, to make a new assessment of the uterus; the other way of behaving, is when, after having repeatedly rubbing the uterus, the erection conforms slowly without reaching the intensity that characterized it in the previous phase. It differs from the previous one, because the local rubbing loses the ability to extend the erection to the rest of the uterus, but the horns kept its tubular shape against some resistance to the touch of the fingertips. This slow erection of the uterus is also characterized because it is not lasting by the mere fact of holding it on the hand, and because it vanishes as soon as we release, indicating that the follicle is in the pre-ovulatory phase, preparing to evacuate. Announcing that the period of the optimum insemination rank has begun. From this moment on, the uterus starts a process during which progressively loses its tubular configuration to the extent that the follicle begins the process of evacuation of the follicular liquid. The muscle that makes up the horns, relaxes to the extent of not appearing as tubular or cylindrical but a liquid membrane that allows the direct touch of the fingers, and when rubbed it erects again, giving the uterus a tubular shape, though clearly showing that it lost size and intensity. At this point passing from locally touching to rubbing, shows clearly that little by little the resistance to the touch of the fingers fades until it is done without any effort, the erection vanishes on hand even when rubbing. This is an indicator that the follicle is in the phase of evacuation, announcing that the animal is still within the optimum range of insemination. Finally, we will describe the appearance of the uterus when it is at the right time to be artificially inseminated with preserved semen from a calf, it is an appearance that differs from the previous because rubbing does not generate an erection or configures a tubular shape, but the horn where ovulation occurred, adopts a flattened, completely flaccid shape. It does not oppose any resistance to the free touch of the fingers. In this phase we can ensure that ovulation has been completed and we have all the chances that insemination concluded in successful fertilization of the egg.

C).—Technique of Manipulation of the Uterus to Deposit Sperm in the Oviduct.

It consists of a series of maneuvers that aim to raise the horns with the inverted tip, in position of penetration, in such a way that the horn can be held and flushed mounted on the applicator of semen. It is based on a series of movements and default turns of the hand and fingers to accommodate the active horn. Each horn has a particular way of manipulation according with the hand used to handle it, however, because most of the inseminators use the left hand, we will explain only the movements of the left hand, for those using the right hand the movements are exactly the same, as if you were seeing through a mirror.

We will start by the left horn, but both start with the correct positioning of the hand on the curvature of the horns. To do this you must first accommodate the thumb above where it starts the external fork, and the index and middle fingers, below the bifurcation, arranged transversely, holding both horns, after you must pull the fingers from below and mount them transversely on the horns, and if it attempted to hug them, and already then, slide the hand following the natural curvature of the horns where it clearly feels that they separate, to then continue with the following:

First, sliding the hand to the left, hook the index, middle and ring finger, on the shape of the horn, positioned to move them, following the natural curvature until the thumb and the index fingers reach the tip or the closest part to it, holding it firmly to pull in direction to his heart, until it manages to draw and place the horn on top of the uterus, then, with a clockwise twist of the hand, leaving it positioned with the inverted tip.

Now, let's explain the move to invert the tip of the right horn. There are two ways to facilitate this operation: The first part is made where you clearly feel the separation between the horns, interpolating the hand with the fingers extended, touching with the palm of the hand the right horn, then, introducing a bit more toward the tip, until you feel it rests on the fingers, so you can push upwards, along the side, until it manages to reverse the natural position of the horn, with the tip up, on top of the uterus, well-off so that it cannot so easily return to its natural position, to give time to take the hand and grab from above the tip, grabbing it firmly between the thumb and index fingers, to pull in direction to his heart, until you are sure that the horn is fully inverted. The other way of manipulating the right horn, is carried out with another series of maneuvers. The first is to embrace the horn sliding the hand until the index, middle and ring fingers, surround the horn, and until the thumb finds space and conforms to get it between the horns; looking to make contact with the index finger, because in this way, closes a gripper that embraces and ensures the horn, conformed in a way that it can slide the hand following the natural curvature where possible to move forward, as close as possible to the tip. Because it facilitates and ensures the success of the next maneuver, which consists first to ensure the site of the maneuver, compressing without pressing the thumb against the index finger, and then make a clockwise turn of 180 degrees. This maneuver has the purpose of accommodating the horn next to the palm at the end of the turn, to hook on the bent fingers, and thus continue the way to the tip, where with the help of the thumb and the index finger, hold firmly to pull towards his heart, until the horn is lifted and accommodate the inverted tip.

Regardless of the horn that is concerned, once you have the tip of the active horn in position of penetration, it is possible to proceed to get the horn on the applicator of semen. Begin by releasing the tip of the horn sliding the fingers down by the shape of the horn in direction to the fork, from the tip backwards, until that hand stays with the index and middle fingers positioned just above and in front of the place where you find the tip of the semen applicator. With a movement in decline, it can insert part of the horn on the applicator, and with a movement of ascent, taking it back up, to insert what is advanced, and thus systematically, repeat these movements until taking the tip of the horn is achieved to give the last movement of decrease to fit the oviduct on the applicator.

D).—Size of the Active Ovary.

Another event that is worth mentioning and which also serves as a tool to define the optimal moment for insemination is the change in size of the ovary during ovulation, due to the release of the follicular liquid. It is one of the easier signs to perceive when we care to keep in mind how was the ovary when the follicle reached its maximum development, since it reduces its size to the extent that ovulates.

### BRIEF DESCRIPTION OF DRAWINGS

FIG. 1 illustrates a graph of the relationship between the moment of insemination and fertility.

FIGS. 2-44 illustrates the physical form of the ovary during several stages of the life of a cow.

FIGS. 45-73 illustrate the steps to follow in the method of artificial insemination in accordance with the present invention.

FIGS. 74-102 illustrate the different techniques that can be used in the method of artificial insemination in accordance with the present invention.

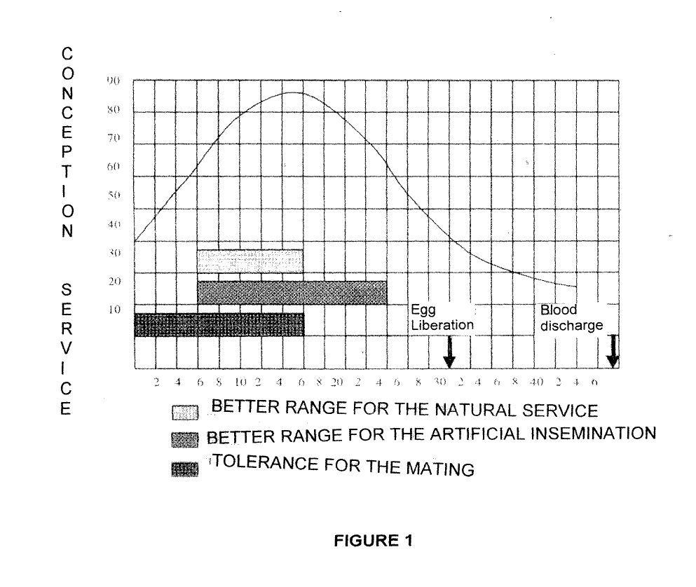

### DESCRIPTION OF THE INVENTION

Before describing the method of artificial insemination that we intend to patent, let's define a set of terms that we will help to understand the methodology:

Reproductive Cycle: The period of time during which a female develops all the physiological processes involved in reproduction, from the development of the uterus' ability to nurture new life to the induction of the estrus cycle to conceive; from the conservation of the state of pregnancy of the cow to the time of calving.

Active Horn: The uterine horn in which the ovulating ovary is located.

Successful Fertilization: Opportune fertilization of the ovum by a vigorous sperm.

Active Follicle The follicle which undergoes the ovulation process.

Ovarian Genotype: Typical genetic stereotype of either a bilaterally or unilaterally operating ovarian system of a cow or heifer. Three types are known in which:
a. The ovaries alternate functions with each new estrus cycle.
b. The ovaries alternate functions with each new reproductive cycle.
c. Only one of the two ovaries works throughout the reproductive life of the animal.

IA: Artificial Insemination.

Successful Insemination Insemination that ends in pregnancy.

Handling by Reproductive Priority: System of grouping cows according to the physiological state of the uterus.

Active Ovary: The ovary in which the ovulating follicle is located.

Bovine Bioreproductive Clock: It is the genetic memory which defines the sexual behavior of the female. It is used to predict and specify the reproductive events of economic importance to the farmer.

Reproductive Health: Status of functional normalcy of the reproductive organ of the female.

Young Heat Detecting Bull: Sexually immature male bovine animal with a strong libido used to detect heat

Heat Detection Bull: Male bovine animal surgically altered or physically prevented not to mate that serves to detect cows in heat.

Healthy cows of heifers: female bovine animals that go into heat and subsequent ovulation without assistance, free of intrauterine infections and diseases.

Optimal frame of insemination: The period of time or window of opportunity during which sperm is deposited in anticipation of imminent ovulation.

Optimal moment for insemination: The most propitious moment physiologically for the successful union of the ovum and the sperm. It is recognizable to the palpation because we perceive that at least ¾ of the follicular liquid contained in the follicle has been evacuated and because it is now possible to touch the bottom of the follicular cavity with the finger and realize that membrane can slip over its entire surface.

The method of artificial insemination of the present invention increases to 100 percent the chances of success to impregnate a healthy cow or heifer in the first attempt, but it requires much more attention from the inseminator while he or she dominates the method with expertise. It is a method that even before the improvements that we claim to present, is based solely on palpation of the follicle to determine the optimal time for insemination, and the deposit of the defrosted semen at the tip of the horn.

The method of artificial insemination of the present invention is based on depositing the sperm directly in the oviduct, just when we perceive in the active horn that the follicle is about to complete the process of ovulation.

The farmer must know that there are marked differences in ovulation time between cows of a same breed and between the various bovine breeds and this, while somehow prevents to recommends or generalize rigid schedules to inseminate a herd, it does let you know that the ovulation of every cow, is a physiological constant, that birth after birth always behave in the same way. Each cow has a defined schedule in the course of his life and this physiological aspect can be maximized if staff and livestock are organized differently from the traditional.

In the method of the present invention, the inseminator must learn to feel the optimal time for insemination, which is not done in the traditional method. The inseminator learns to feel the ovary, but especially the horns, to determine the optimum time which he must enter the sperm in the oviduct. Another difference of the method of the present invention in relation to the traditional method is the place where the semen is deposited. The prevailing idea, in the traditional method, that it is not necessary to place the semen deeper than in the body of the uterus because sperms are capable of moving to the ovum by themselves, it is believed that placing the semen deeper in the body of the uterus, can cause intrauterine damage.

The traditional method does not take into account the capacity of sperm cells to navigate from where they are deposited up to the point where they are needed under intrauterine conditions which are not the most favorable. Also not taken into account, the necessary energy of the sperm to fertilize the ovum, successfully likewise not taking into consideration the cow's particular biological rhythm of ovulation, hence the failure, and hence the difference with the method of the present invention, because all these aspects are involved and successfully combined in the new procedure with the deposit of the semen in the oviduct.

The method of the present invention recommends that staff be trained to respond comprehensively to an efficient program of artificial insemination. In this aspect, the method of the present invention particularly recommends that the inseminator or the inseminating team becomes familiar with the cows, not only to check for heat and inseminate them as in the traditional method, but also to familiarize with some natural biological processes of delivery and post partum of the animal. It is necessary somehow, that the inseminator will be able to palpate, to determine that the cows are healthy and are suitable for reproduction, for not having to waste time or money on sick cows, nor in cows sexually inactive.

We recommend gathering together all the animals that are going to receive the first servicing into special areas where the inseminator or the insemination team can closely observe and care for the healthy cows and heifers while they are in heat, and from where they can easily move the cows to the particular production group that corresponds to them.

It is important for the inseminator to familiarize with the herd. The inseminator should take advantage of the habits of livestock in the field. The inseminator must revise the livestock before dawn, to observe the cows which entered in heat during the night, because, they are usually standing before the routine of grazing. Generally the most difficult time to observe the herd for heat is usually at sunset, since it is necessary to see them when they begin to gather together to rest and to wait for nightfall to be sure of detecting any animals in heat.

### Detection of Cows in Heat

Heat is the physiological condition in which a cow or heifer manifests uncontrollable sexual desire. It is the moment the cow or heifer allows any animal to mount her in order to satisfy her desires in this ardent period of sexual activity. In some cows, heat can last six hours or less, and in others up to 30 hours or more. On average, it lasts between 12 and 18 hours in European breeds and not as long in zebu breeds. In this condition, also called estrus or sexual receptivity, the cow accepts being mounted not only by the bull, but also by other cows. This does not indicate a hormonal or emotional disorder.

This sexual characteristic of cows is what indicates to us that the animal is ready to copulate and conceive. Detection of heat is easy because the cows get excited, and even the most frivolous have sexual manifestations in a very short period of time during which a large number of them will only mount and accept being mounted once in a while. It is necessary to identify the individual cow's habits because every year, they will always behave in the same way, and it is important to know their habits to avoid so-called “silent heats” which go undetected because they are manifested discreetly. Observation of the sexual behavior of each cow will indicate whether heat is in the initial, intermediate or final stage. One should learn how each individual cow demonstrates that it is in heat, and how many hours that the animal remains in heat year after year, because every time that the animal goes into heat, it will always manifest the same behavior.

The first phase of heat is characterized by the madness provoked by the waves of amorous desire surging through the cow's body, the result of a hormone called estrogen. When this starts, the desperate cow begins, if there is no bull nearby, to search among her companions for someone who can help satisfy her, trying to stimulate them by smelling and licking their genitals. At the same time trying to stimulate herself with a singular, rhythmic hip movement before trying to mount others. In this first phase of heat, the cow shows a restless twitching of the ears and walking among her companions enticing them to mount her until she gets one of them to do so. It is a state of restless activity that the cow immediately tries to mount any cow that has mounted or attempted to mount her. In this way she continues, until finally, she allows herself to be mounted and does not move, remaining totally motionless until the one that has mounted her decides to get off.

If the cow does not get any of her companions to mount or if the cow is alone, the cow moves by the perimeter of the fence mooing desperately to draw the attention of some animal who wants to sooth her sexual yearnings. When the cow that is just going into heat meets another that is already in full heat, the cow going into heat behaves like a jealous bull; pushing away any other cow that approaches her companion, showing an aggressive sexual behavior characteristic of the beginning of heat.

The second phase of heat, the intermediate phase, only differs from the first in that the cow no longer shows its intense desire to mount any cow that passes by or a cow which she has just mounted. In this intermediate stage, which is of the longest-duration, the cow still mounts other cows and wants to be mounted, but is no longer the desperate animal consumed with desire that lives and dies for love. She simply remains with the group that she has formed around her for her satisfaction; mounting or being mounted at ever longer intervals. Then we have the final phase of heat, which is the most difficult to determine because the cow might continue mounting, but begins to lose the desire to be mounted. In this phase, she no longer stands as still for as long as her companion or “heat-detector bull” would like. The cow no longer displays a craving to be mounted and tries to distance herself from the one that has mounted her but not from the company of her whole comfort group or her companion. This behavior continues until the cow finally rejects being mounted, showing that she no longer desires copulation but still wants to remain with her companions, who she will try to mount again every once in a while. Then suddenly, the cow receives a hormonal discharge that puts an end to this sexual scandal. She isolates herself entirely from the rest of her group, and if she has no place to hide, keeps her companions at a distance by pushing them away or lies down so that she can't be mounted thereby demonstrating the end of the hormonal event. The method of this application is based on knowing the precise moment when the cow rejects mounting, the end of this particular sexual stage. This is when the countdown in hours begins, before the cow's bioreproductive clock determines the optimal moment for insemination. This period is, as is in the case of heat, a constant which is peculiar to each cow.

Finally, let us end by pointing out that to maintain a successful insemination program; we must identify heat in all of our cows. This is a relatively easy task if we concentrate on those cows that are going into heat and will receive the first insemination service into a compact area that can be comfortably and easily observed and if we establish a strict routine of checking for heat and a suitable follow-up program.

### Management of Cows in Heat

The most important thing we have to do is to isolate the cow in heat in an area of our facilities where it can be comfortably observed, where we can periodically check its condition, and from where we can, under any weather conditions, steer it without difficulty to the cattle chute for examination and/or insemination. To do this, it is essential to keep the following in mind:
1. If the cow in heat is kept with cows not in heat, without a heat-detection bull, you must isolate the cow and herd her to the observation area, along with at least one of the cows that has been mounting her. But note that it is necessary to provide reinforcement, because the cow that is not in heat gets bored. Preferentially, provide a young heat-detection bull that you know beforehand has an aggressive sexual impulse. This bull must always be kept separate from but within eye and olfactory contact with the cow in heat. This allows you to determine whether the cow is still in heat because she will stick close to that part of the fence where the bull is standing and because it is apparent that the bull will immediately try to mount her when they are brought together. This way you will not have to waste time guessing whether the cow in question is still in heat or not.
2. If more than one cow is in heat, with or without a heat-detection bull, the cows in heat should be separated from the rest of the herd and moved to the observation area. Note that if you have only two cows in heat, you have to watch carefully in case one of them goes out of heat, because it is very common for a cow to lose interest in mounting its companion when one of them is no longer in heat. When this happens, it is again necessary to have a heat-detection bull in reserve in order to be able to continue checking whether the cow in question is still in heat, especially when observations are being made under adverse weather conditions.

### Detection of the Ovulating Ovary

Being able to identify the ovary that has the follicle that will ovulate, and to determine the most propitious time to deposit semen are the two most important technical aspects of the new method to consider when training the inseminator or insemination team. It is primarily these two practical technicalities that determine whether or not a cow gets pregnant on the first attempt at insemination. To identify the ovary with the follicle to ovulate, it is necessary to know how the ovary is, how the follicle is and what physical changes take place in the ovary and the follicle from the end of heat to the end of ovulation.

Before we start we have to comment on a series of technical concepts and events that should be understood theoretically in order to correctly interpret what is happening to the cow and what we have to do with it once she goes out of heat.

Firstly it is necessary to understand what the reproductive organ of the cow is like after heat ends, what structures it consists of, and what each of the components and parts in which the uterus is divided is like in order to study it. There must be a clear idea of what has to be inspected when examining a cow to determine where to inseminate it.

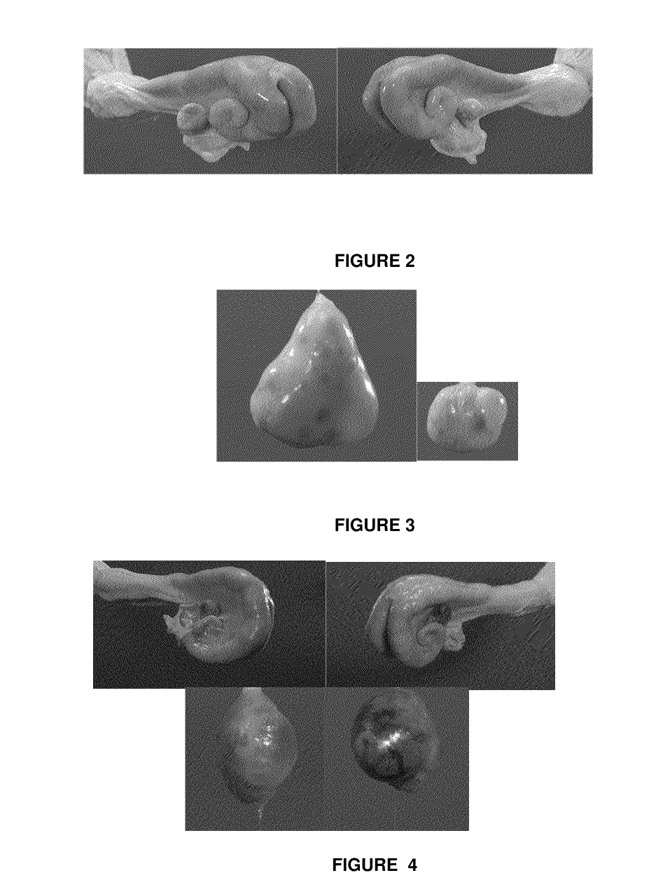

FIG. 2. Shows a view of the uterus from the left and right side respectively as located inside the animal. The images show cervix 10, uterine horn 20, external bifurcation of the horns 30, oviduct 40, right ovary 50, corpus luteum 60, Graaf's follicle 70, left ovary 80 and primary follicles 90. The images show the whole structure of the uterine horns perfectly 30, from where they start together at the cervix 100 (the zone clearly identified by the change in color), to the tip where semen is deposited 110.

Secondly, it is important to know why a cow or a heifer goes into heat and while this is not a reproductive physiology course. We must focus on some fundamentals that can be confirmed in practice. Any cow or heifer goes into heat because one of its two ovaries develops a structure similar to a liquid-filled blister, which releases a hormone that sexually arouses it and makes it accept copulation. This blister is technically called Graff's follicle 70, and is the structure that hosts and matures the female particle of procreation, the ovum. The Graff's follicle 70 indicates when we have to deposit the semen, because in them occurs the physical changes that gives us a guide to determine when to inseminate the cow. These changes in the follicle are easily perceived by palpation. These changes always occur after heat ends and always take place over the same period of time. Thus these changes become the most important physiological constant of artificial animal reproduction because it is upon them that the timetable by which each cow must be inseminated depends. It is the natural biological schedule of each cow. In practical terms each cow throughout her reproductive life has a natural and constant timing that is particular to it alone, and varies only with the season.

That is why before continuing with practical matters we must turn to yet another very important theoretical aspect, the estrus cycle. The point of understanding the theoretical aspect is to know that in each cycle, a follicle grows and ovulates 70 and in the place where it grew and ovulated, another structure, the corpus luteum 60, forms, develops and takes its place to perform another function. The corpus luteum 60 is born after the hole left by the follicle 70 is empty and closed. The corpus luteum 60 grows slowly as the hours and days go by until it completely fills the follicular cavity that gave it life. If the animal is not pregnant, the corpus luteum 60 also shrinks slowly, until it disappears at the end of the cycle. When the ovulation of another follicle terminates which formed and developed in the other ovary, while the corpus luteum was growing and being reabsorbed.

Therefore, the estrus cycle can be defined as a natural biological cycle that induces reproduction by the manifestation of uncontrollable sexual desire within the cow and is caused by a physiological reproductive mechanism that ensures the release of the ovum just after a cow or heifer has copulated with a bull. In most animals the cycle is usually constant, with a few irregular exceptions; but in all cases the cycle marks the beginning of a new period of heat that indicates the start of a new estrus cycle, in which the functions of the ovaries alternate. The ovary that in the last cycle developed a follicle, in the new cycle will develop a corpus luteum, while the other ovary develops a new follicle.

The aim here is to give you a frame of reference with which to find the right time to palpate the cow in order to determine which ovary has the active follicle and to determine the schedule for possible insemination. In most cases you will encounter cows and heifers that have gone into heat more than once before being inseminated, normally referred to as cycling cows—which means that when palpating their ovaries at the end of heat, they contain besides the active follicle, the corpus luteum which is in the process of disappearing and being reabsorbed in their ovary.

In practice it is very easy to identify by size which ovary is which and what each one does. It is very easy because at the end of heat, the bigger of the two ovaries is the one we are interested in—the active follicle.

The ovaries can be of wide variety of shapes and sizes. FIG. 3 shows the ovaries of two animals that have gone into heat. Note especially that the follicle of the bigger ovary is much larger than the entire ovary of the other cow. Despite the different sizes, however, both ovaries and both follicles are equally suitable for reproduction.

Cows with Ovaries that Alternate Functions with Each Estrus Cycle

This ovarian genotype is characterized by the way the two ovaries function during the estrus cycle: one maturing a follicle and the other maturing and/or reabsorbing a corpus luteum, so that with each new cycle each ovary assumes each of these functions in turn as long as the cow does not become pregnant. We can easily identify which ovary is which at the end of heat by the differences they manifest in size and/or shape, and sometimes only in consistency. Difference in size because the ovary that has the active follicle, is often bigger than the one with the corpus luteum in re-absorption; difference in shape because the active follicle is a round structure that gives the ovary its round appearance where it is located, while the corpus luteum in re-absorption has a characteristic bump which generates a rigid eruption on the surface of the ovary that makes it look deformed. Difference in consistency because the palpable surface of the active blister-shaped follicle is completely smooth and elastic, and is a flexible membrane that absorbs the pressure from the finger that probes it, while the ovary that is reabsorbing the corpus luteum feels hard to the touch over its whole surface.

This ovarian genotype is the most common, and is often the cause of confusion among inseminators who are just learning the method. This usually happens when they encounter ovaries of similar size, because they do not take into account their structure or consistency. Inexperience practitioners do not have mental maps of reference for what they may encounter.

FIG. 4 shows ovaries of the same size. If one palpates this cow, one would notice immediately that the ovaries are the same size. Even an expert inseminator wouldn't note in which ovary is the active follicle. A shape analysis will help you identify the structure of each ovary; the active follicle of the right ovary is easily noticed by its roundness and smoothness, while the corpus luteum of the left ovary can be recognized by the hardened deformation on its surface. The difference between the two is the consistency, the smoothness of the surface and the elasticity of the active follicle, versus the non-elasticity of any part of the ovary with the corpus luteum in regression.

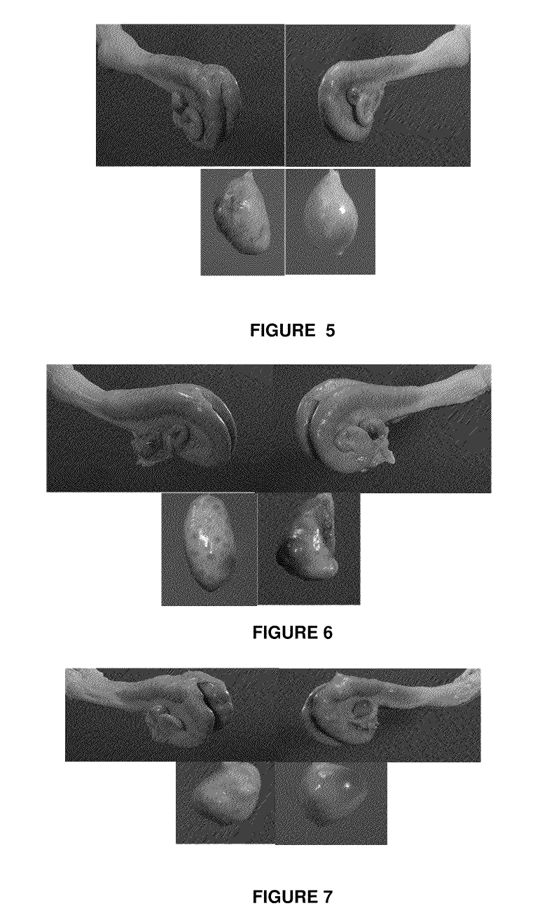

FIG. 5.—in this photo we see the rare case in which the inactive ovary is only of a slightly larger than the active one. In this instance the inseminator will have to disregard the larger ovary on the right. Because the protuberance of the corpus luteum in re-absorption has not emerged and the ovary has a round appearance, the ovary is hardened over its whole surface, and doesn't show a sign of elasticity anywhere which indicates that it lacks follicular blisters. On the other hand, the left ovary, besides being smaller in size, and round in shape, has an elastic consistency due to the presence of the active follicle.

FIG. 6 shows the triangular structure of the ovary containing the corpus luteum in regression, a difference which is made obvious because the protuberance of the corpus luteum is located at the top of the ovary. In these cases in which the ovaries are roughly similar in size but very different in shape, beginning inseminators can focus their search on the active follicle, by disregarding the ovary with the corpus luteum because of its irregular shape and the absence of any roundness anywhere on its surface, and by concentrating instead on identifying in the other ovary all the typical features of the active follicle: its characteristic roundness, the smoothness of its surface, and elasticity of its outer membrane.

FIG. 7 In this last set, the two ovaries are similar in size, but of a marked difference in consistency. The right ovary has a hard appearance, with a bump on the surface that appears equally rigid, while the left ovary displays in one of its parts a clear, round, smooth blister, which feels resilient and elastic to the touch allowing the fingertip to sink into it and resuming its original shape once pressure is released. Despite the special nature of the preceding cases, what is the most common in cows in which both ovaries work in alternate cycles is that there is always a clear, palpable difference between the two ovaries, most often in size, with the active ovary always being the larger of the two, as is confirmed by the following pictures which illustrate how easy it is to differentiate one ovary from the other.

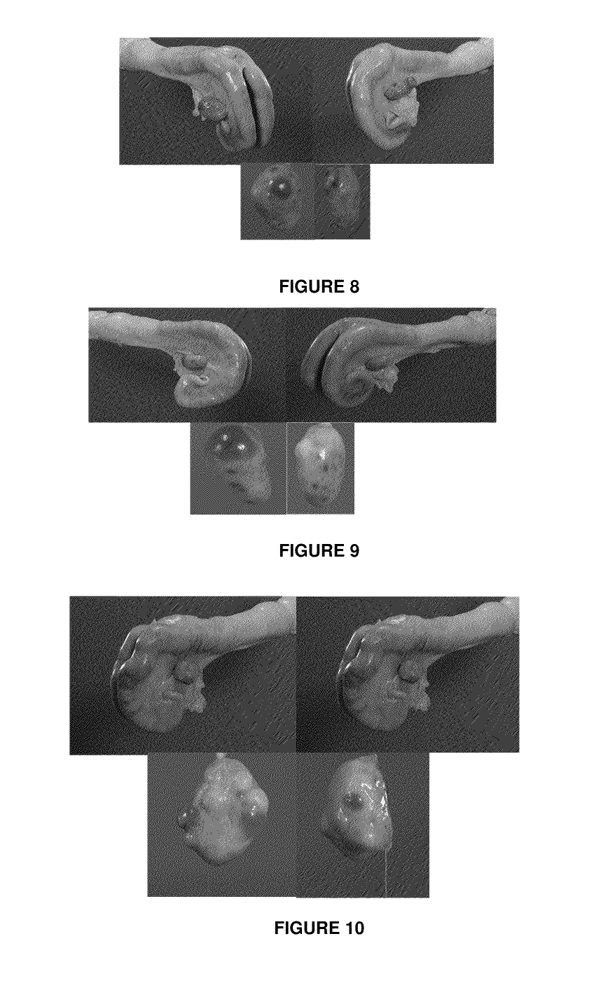

FIG. 8 This case shows a typical example from cows in which the two ovaries alternate functions with each new estrus cycle, the difference in size and structure is evident. The inexperienced inseminator must focus his search for the active follicle to the larger ovary, probing until he finds that part where he can feel the characteristic roundness of the follicle a smooth blister with an elastic consistency on the surface of the ovary. Likewise, he must make sure to disregard the inactive ovary, which has the corpus luteum in re-absorption, because of the rigid bump that protrudes from its surface and for the absence of any roundness of the ovary which would indicate the presence of an active follicle.

FIG. 9 shows the roundness and softness of the end of the right ovary where the active follicle is. The difference in the size of the ovaries is the main indicator of which is the active one; although the left ovary can be disregarded because it is slightly flat, without any perceptible follicular blister, and has a rigid protuberance on its surface.

FIG. 10 clearly indicates that the animal has been cycling without conceiving for a long time. Notice the irregularity of the surface due to the presence of the corpus luteum and the corpus albicans in both ovaries. Note also that both have follicles, and that there is one that predominates over the others. In this case, the inexperienced inseminator in his search for the active follicle must focus on the size of the blister.

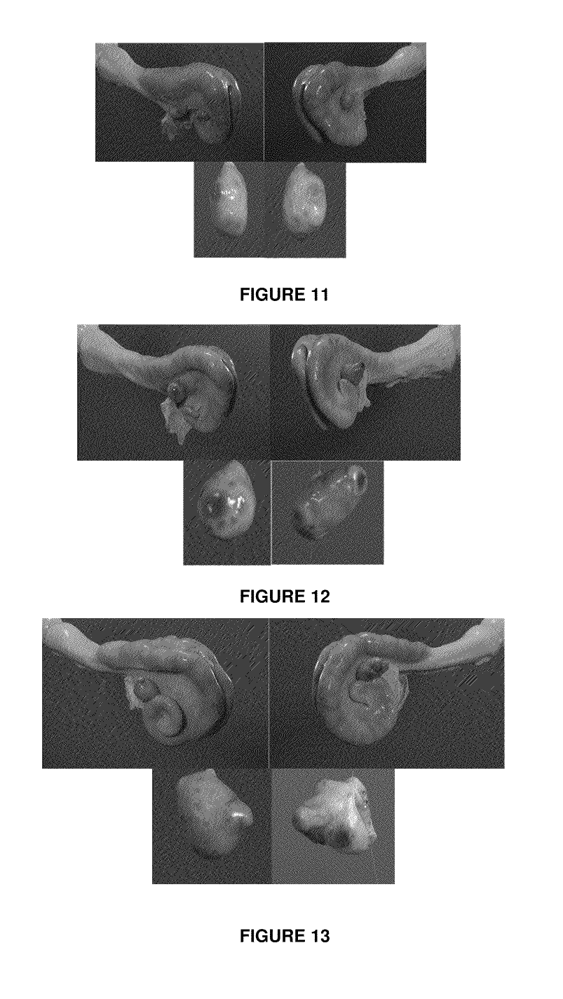

FIG. 11 shows ovaries of very similar size and shape. One can distinguish the active ovary from the inactive because the ovary with the active follicle will always display a round area that characterizes this ovarian structure; and in this case, where both have a bump that emerges on the surface, the difference is in their consistency. While the bump on the right ovary feels soft and elastic and absorbs pressure applied to it by the probing finger, the bump on the left ovary is rigid as is the ovary itself.

FIG. 12 shows the difference in structure between the two ovaries. First, if one touched the right ovary would notice the roundness which makes up the active follicle, with its smooth surface and its soft, elastic consistency. Likewise, if one could feel the ovary on the left one could eliminate it from consideration because of the unmistakable irregularity of the crater-shaped corpus luteum. The novice inseminator should always feel both just to make sure, because the left ovary has a small secondary follicle; this is not enough to create the typical roundness that distinguishes the follicle of the other ovary.

FIG. 13 shows a very special and rare case of a classic example of segmented ovaries. It is very easy to distinguish which is the one that will ovulate, because, even though the ovary on the right has an oval appearance, it appears hard, with the protuberance that is characteristic of the corpus luteum and without any perceptible follicular blister. The ovary on the left, on the other hand, is clearly divided, with one of its parts hard and the other soft and totally elastic.

Cows with a Single Functional Ovary for Each Reproductive Season

As for cows that activate a single ovary for every reproductive season, the main indicator of the active ovary is the difference in the ovaries' relative size; the active ovary being at least double the size of the inactive, making it easy to distinguish between the two precisely because of this very marked difference.

This ovarian genotype is characterized by the fact that the ovaries work independently of each other without help from the other and is responsible for preparing the uterus to be able to house a new life, induce the estrus cycle to conceive, preserving the cow's pregnancy and inducing parturitium.

This genotype is the second of the cases that will be presented, nevertheless is the ovary that remains latent and enters into activity suddenly. It is not yet known what it is exactly that triggers or inhibits this activity. In this ovarian genotype, note that the active follicle and the corpus luteum are always found in the same ovary and it is precisely this peculiarity that accounts for the fact that the size of the active ovary is always double of the inactive. Also note that such structures can be developed indiscriminately anywhere in the ovary.

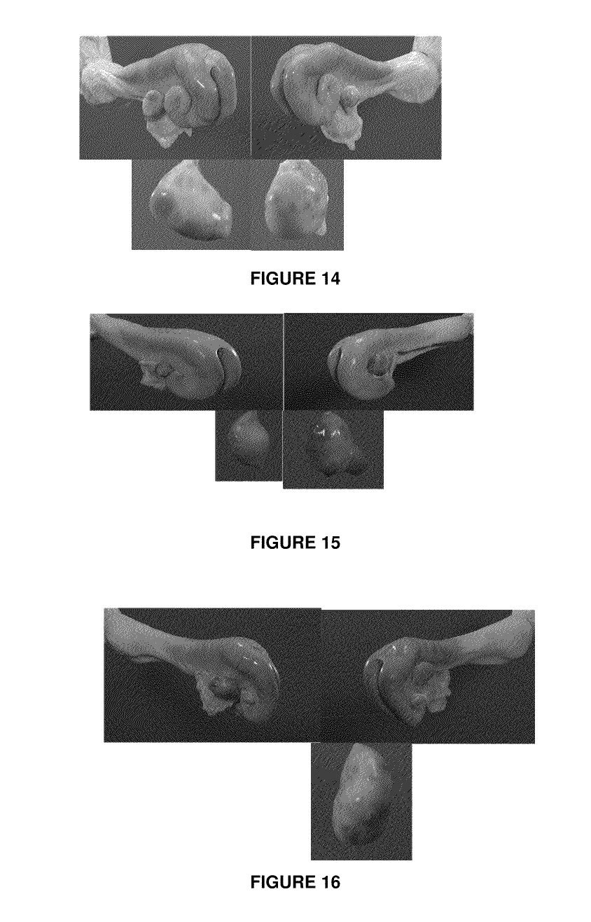

FIG. 14 shows a heifer in which we can see that only one ovary is working during the reproductive season because the active follicle and the corpus luteum are in the same ovary. The other ovary, although it has secondary follicles, does not show any scars of a regressive corpus luteum and is half the size of the active. Note in the ovary on the right the clear roundness of the active follicle at one end, and the hardened bump of the corpus luteum in re-absorption at the other.

FIG. 15 shows a cow in which a single ovary works during the reproductive season. Note that it is the ovary on the left that has been in full activity, because it shows an active follicle, the presence of a corpus luteum in regression, and a clear scar of the corpus luteum from the previous cycle. This indicates that it has been through previous cycles using the same ovary. This is why it is double the size of the latent ovary—in this case, the one on the right. It also clearly manifests the scar of a corpus albicans and the presence of growing primary follicles. It is also characterized by its hardened consistency.

FIG. 16 this case is another typical example of cows in which just one ovary works during each reproductive season. The size of the ovaries is the obvious difference when identifying and choosing the active ovary. Two follicular blisters and a corpus luteum in regression are the structures that make it double the size of the latent ovary, which is characterized by the scar of an old corpus albicans and the presence of growing primary follicles.

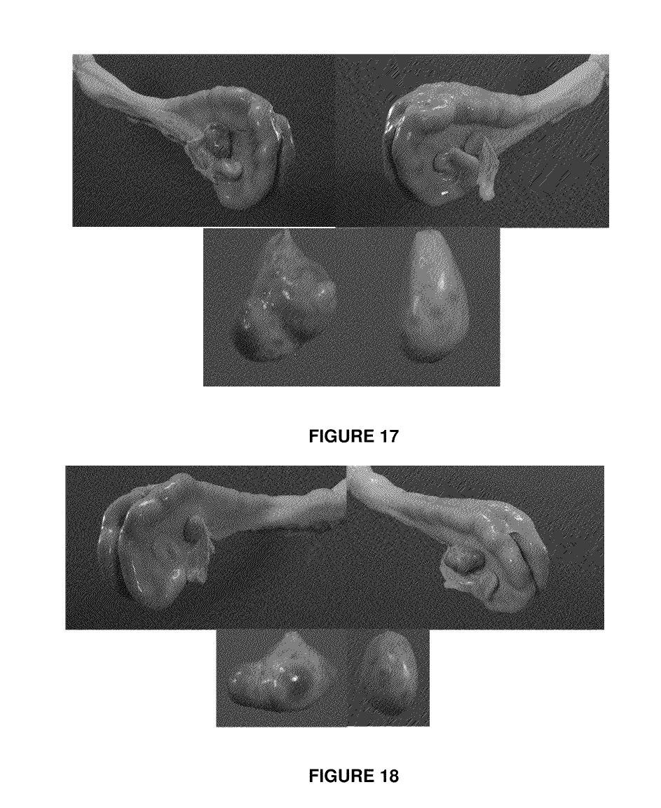

FIG. 17 shows another case in which only one ovary works during each reproductive season. Notice that the functioning ovary is double the size of the other, and appears segmented, because of the position in which the active follicle and the corpus luteum in regression have developed, while the latent ovary, though lacking any corpus albicans reveals the presence of primary follicles that indicate it will become active someday.

FIG. 18 shows one of the many shapes the active ovary can assume when it is developing a follicle and at the same time reabsorbing the corpus luteum, but it is always the size and the irregularity of these shapes which typify and characterize this ovary, especially in the many cases where the scar of the former corpus luteum persists. The inseminator should focus his search on the ovary that is double the size of the other, on the follicular blister and the roundness that characterizes it, the smoothness of the membrane that composes it, and the elasticity that distinguishes it from the rest of the hardened structures on the ovary surface.

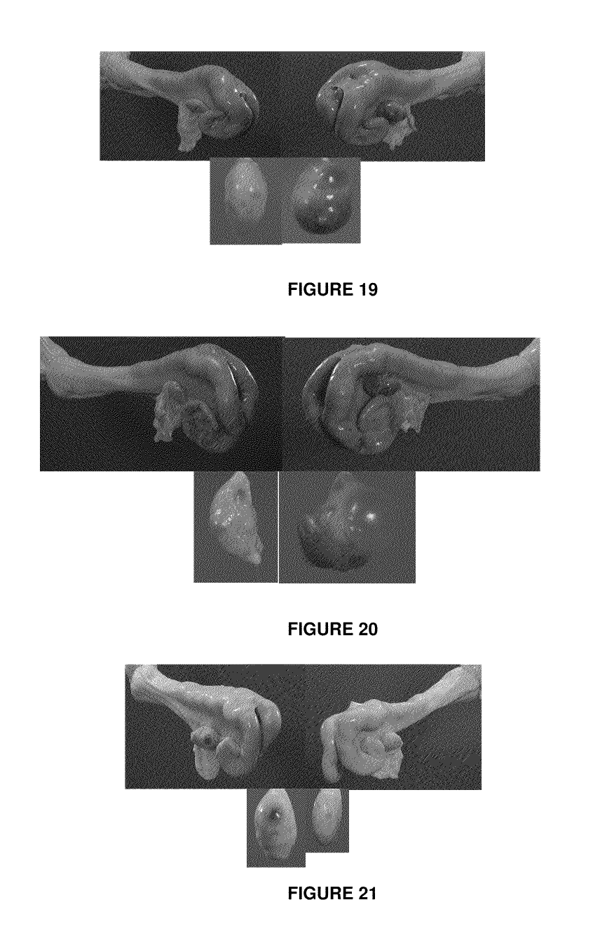

FIG. 19 this is another case where only one ovary works during each reproductive season. It reveals an active ovary of normal appearance clearly showing the protuberance of the corpus luteum in regression plus two follicular blisters that make it grow to double the size of the latent ovary. On the other hand the other ovary looks semi-flat, of a hardened consistency with primary follicles none of which are growing.

FIG. 20 this case shows only one ovary works during each reproductive season, is identified by the size of the left ovary's follicular blisters, and by how easy it is to determine the optimal moment for insemination when it decreases in volume during ovulation. The evacuation of the follicular fluid can be seen and felt by the change in size of the ovary because as ovulation proceeds, the membrane collapses within the follicular cavity.

FIG. 21 shows a cow in which only one ovary works during each reproductive season. It is shown here to illustrate the follicle just emerging on the surface of the active ovary without displaying the roundness that typically characterizes these blister-like structures. It can be detected by the elastic consistency of the follicle surface because it is the only part of the ovary that isn't hardened and where the probing fingertip can sink into it upon applying pressure. In this case the inseminator must first eliminate from consideration the latent ovary by studying its form and consistency and taking note of its flattened shape and the hardness that typifies this kind of ovary. It does not have any rounded areas that make you suppose the presence of a follicle.

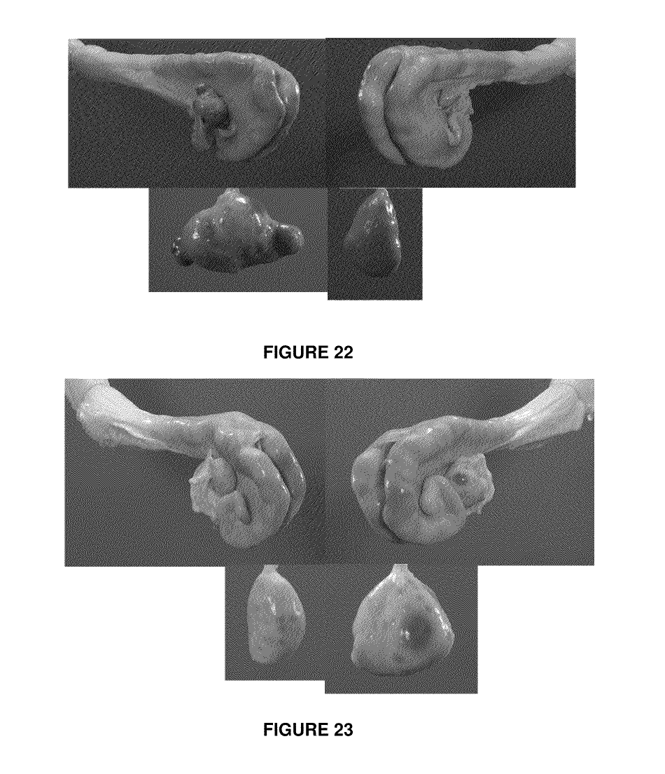

FIG. 22 this case showing a vast disparity in size and shape between the ovaries is also due to the physiological structures of the active ovary. We see very clear signs of the estrus cycle in the same ovary: the unmistakable irregularity of the ovary from the presence of two hardened luteum bumps and a round follicular blister of a soft, flexible consistency. On the other hand, the normal appearance of the ovary on the left looks hardened, without any follicular blisters but with a rigid eruption the corpus albicans which indicates that this ovary has previously been functional, which means that it is now in a latent state.

FIG. 23 In this case only the left side is working, note the clear, rounded structure on the ovary's surface that makes up the active follicle, because it is at the top of this structure where you can feel the elasticity when trying to sink a finger into it to confirm the follicle's presence. Also note that what remains of the corpus luteum in regression is a small bump, barely perceptible to palpation. As for the latent ovary, it is very easy to eliminate it from consideration because of its semi-flat appearance and hardened consistency, and because of the lack of roundness in any of its parts due to the total absence of follicle blisters.

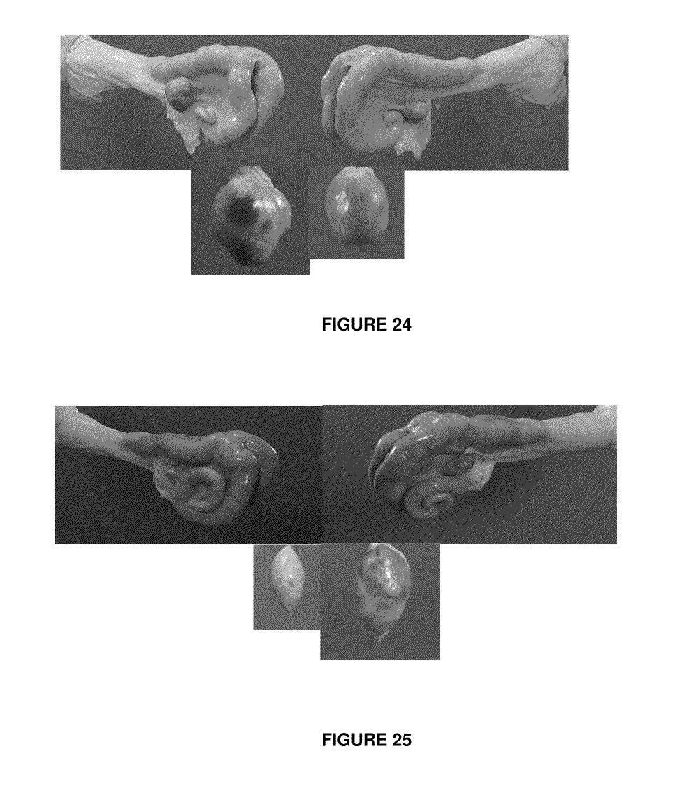

FIG. 24 this case of ovaries that function by reproductive season is exhibited here because the active ovary shows three aging corpus luteum scars in addition to the active follicle. This is a case in which the follicle does not protrude above the surface of the ovary; the inseminator has to identify it by looking for some part of the surface showing elasticity, but also has to make sure that the other ovary is not the one he is looking for because while it has a rounded appearance, it also has a hardened consistency, with a barely perceptible scar, and does not manifest any elasticity anywhere.

Cows with a Single Functional Ovary Throughout Life

This ovarian genotype is the most unusual of all, and is characterized by the development of only one ovary, which is responsible for carrying out all the female physiological and reproductive processes throughout the cow's life. The other ovary never grows or even develops primary follicles, is sterile, and is recognizable because it is at least three times smaller than the active one, and its palpable surface is a hardened mass. This ovarian system is the easiest to distinguish because of the great difference in size between the ovaries, a fact especially worth noting, because if this kind of cow is used for embryo transfer it should not be inseminated in the horn that has the sterile ovary

FIG. 25 shows a typical case of ovarian genotype in which only one ovary works throughout the animal's reproductive life. It is the least frequent but, at the same time, the easiest to identify, thanks to the disproportionate difference in size between the ovaries. The functional ovary is at least three times larger than the sterile one. Note that the sterile ovary is smooth and lacks any growing primary follicular blisters. When it is time to inseminate cows of this genotype, note the hardness that characterizes the sterile ovary.

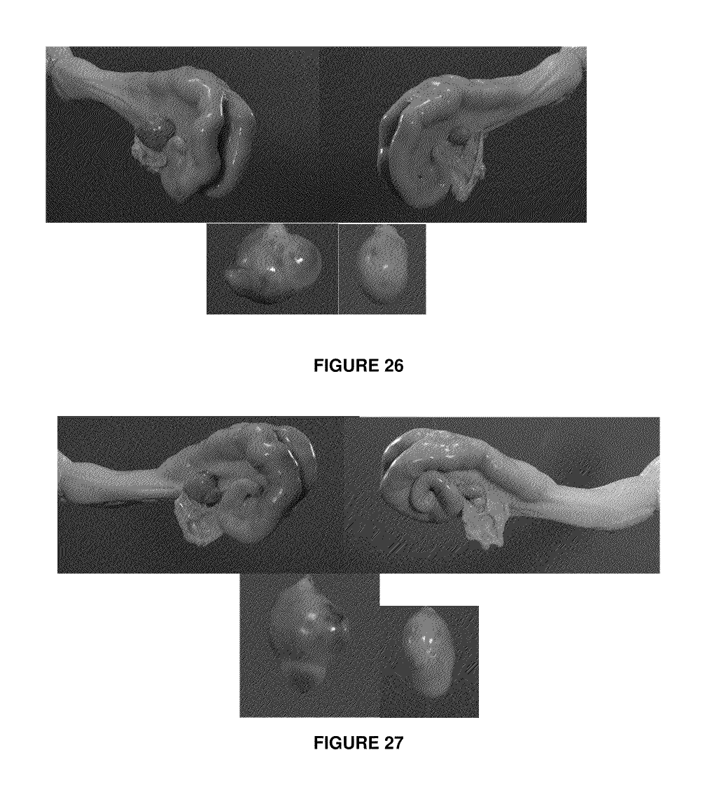

FIG. 26 shows another typical case of one active and one sterile ovary. It shows how easy it is to identify the functional status of the ovarian system by the differences in size and consistency of the ovaries. The left ovary is sterile, as evidenced by its size in relation to the active ovary, but above all by its hardness and the lack of growing primary follicular blisters; while the active ovary is characterized by the protuberance of the corpus luteum in regression, and by the clear, round blister that forms the active follicle. It gives the ovary the characteristic feature of a round-looking area which feels smooth and elastic enough to allow the probing finger to sink into it.

FIG. 27 shows a case very similar to the previous one, and it is the most common form in which this ovarian genotype manifests itself. If you compare it to the previous case, you will realize that the only thing that changes is the position of the corpus luteum and the follicle.

Heifers Going into Heat for the First Time

With animals that are going to go into heat the first time, it is important to mention, apart from the ovarian genotype that characterizes them, that most of the time, more than one follicle is activated in each of the working ovaries. Therefore, heifers of the ovarian genotype in which ovaries alternate functions with each new estrus cycle will develop mature follicles in both ovaries, which appear to be of a similar consistency, and usually of the same shape, but of different sizes. The inseminator needs to identify the active follicle, and the easiest way to do this is to concentrate on the larger ovary because as you can see in the following series of images, the size of the ovary that will ovulate is almost twice the size of the other.

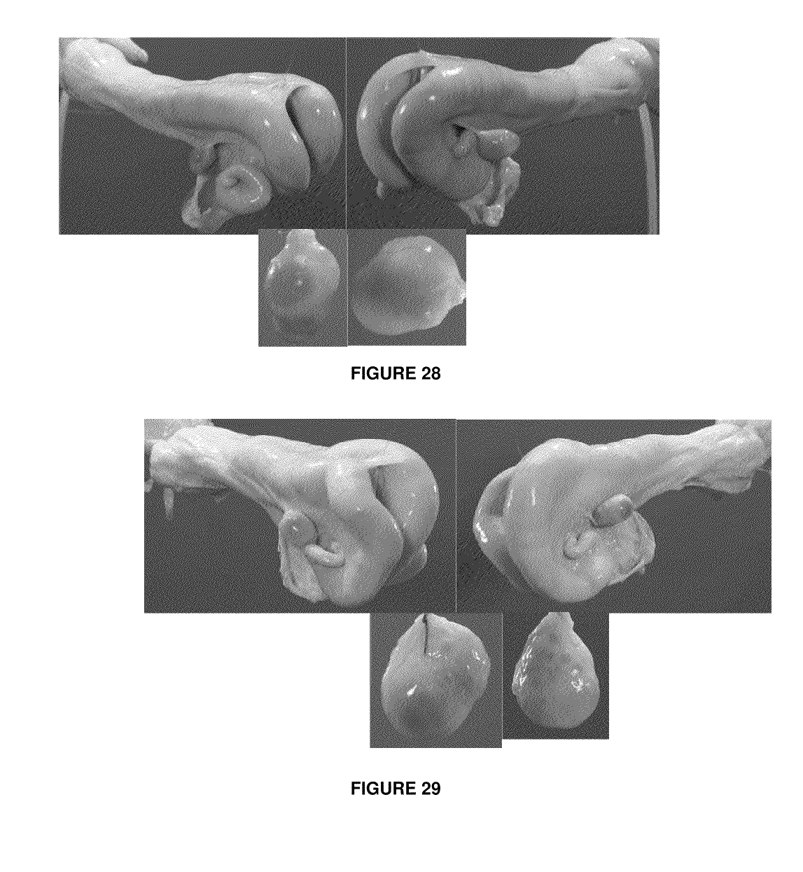

FIG. 28 shows a heifer going into heat for the first time. The features of the ovaries are an indicator of the ovarian genotype that characterizes them. In this case, the presence of very well-developed follicles in both ovaries indicates that they alternate their function with each new estrus cycle. The inseminator can be guided by the size of the ovary in order to discern which one has the active follicle, and then search in that ovary for the larger follicle to determine where ovulation will take place

FIG. 29 shows very well-developed follicles can be observed in both ovaries indicating the genotype to which they belong. Notice the great similarity between the ovaries and their follicles. The inseminator should know that both could ovulate, although his attention should be focused on the larger ovary until ovulation, making sure he palpates the other ovary, as well, to determine whether he should inseminate both horns or not.

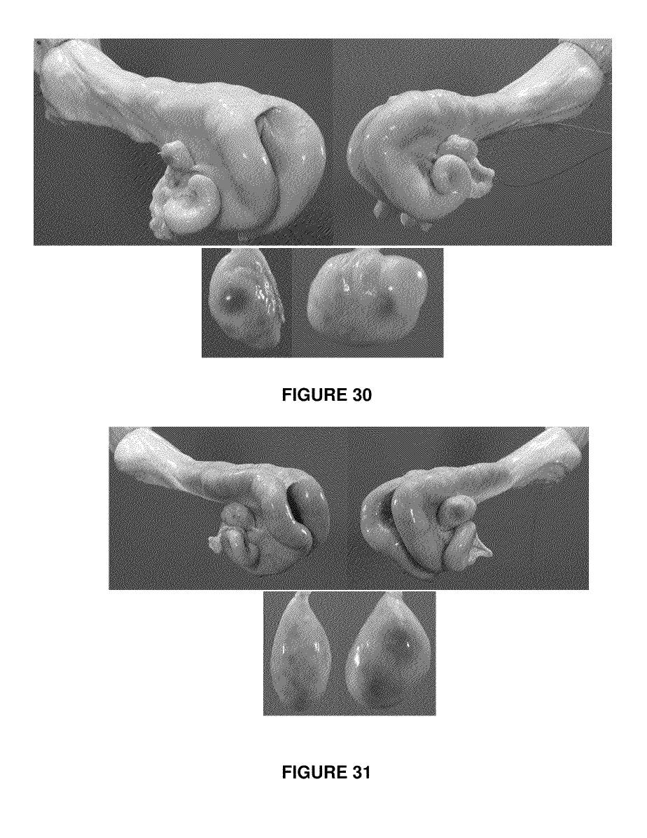

FIG. 30 this case of first-heat is shown here so that the inseminator has a better picture of cases of this ovarian genotype. Notice that the ovaries are very similar in size and shape, as well as the presence of very well-developed follicles in both ovaries, although the obviously larger left ovary is where ovulation will occur.

Regarding heifers with that ovarian genotype in which functions alternate by reproductive season, a somewhat different functional concept applies, because the active ovary is usually only double the size of the latent ovary and quite often also develops two follicles large enough to be detected by touch. In practice this does not present any obstacle to insemination, because whatever ovary that begins to ovulate is going to change in size and shape once it begins to evacuate. On the other hand, the latent ovary is usually half the size of the other, and follicular activity, if it can be perceived, is reduced to stimulating the growth of the primary follicles unless these are awakened from that state.

FIG. 31 shows the size of the ovaries, because this is the clearest way of differentiating them in heifers going into their first heat. Note that the follicular blister is what causes the active ovary to grow to double the size of the latent ovary, which can be disregarded not only because of its size, but because of its hardened consistency and the presence of growing primary follicular blisters which often can't be perceived by palpation. Nevertheless, the size of the ovary and the proactive primary follicular blisters are indicators that they will begin functioning sometime.

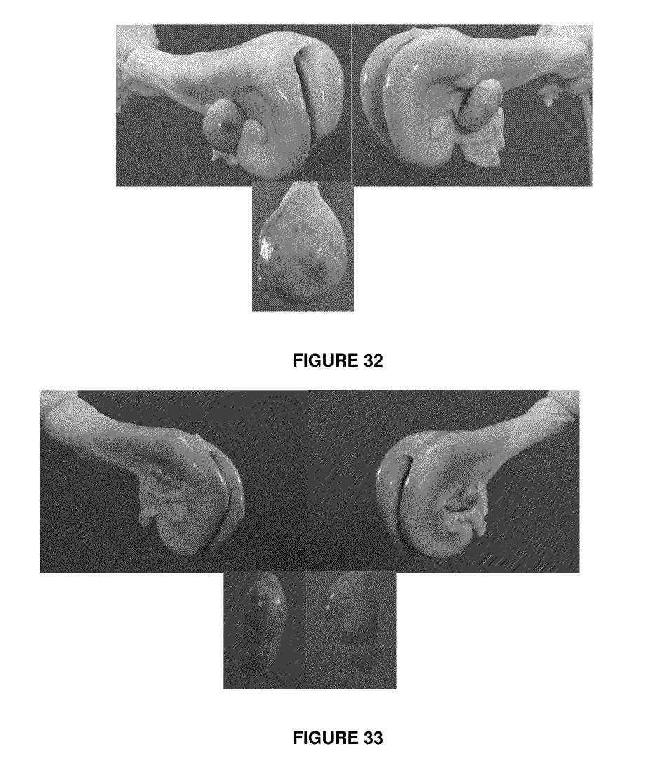

FIG. 32.—shows a case very similar to the previous one, except that here the active ovary is the one on the right, and the proactive primary follicular blisters of the ovary on the left are obvious and easy to perceive by running the finger over the surface of the ovary. Note the large size of both ovaries.

FIG. 33.—Note here the unusual shape of the ovaries, especially the one on the left which shows clear evidence of what will be a segmented ovary as soon as the corpus luteum develops and a follicle develops in the other segment, assuming, more than once when the cow is in actual heat. Note that the ovary on the right has proactive primary follicles that can be palpated in order to specify the ovarian genotype.

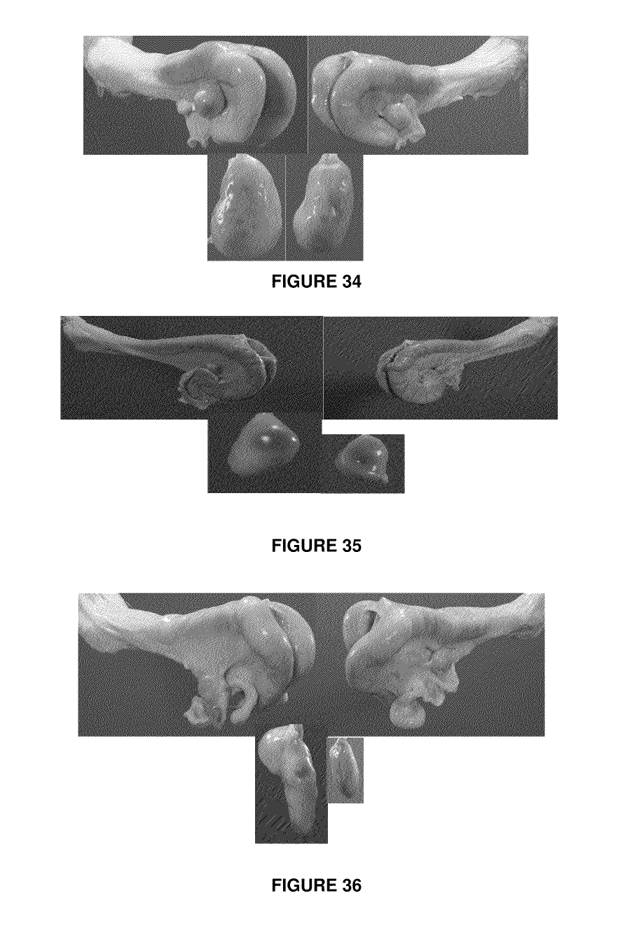

FIG. 34.—In this case, as with others of the same ovarian genotype, it is very easy to differentiate the active ovary from the inactive one by their differences in size and consistency. Note the presence of the only mature follicle in the ovary on the right, which is very easy to detect by its large palpable surface, and in which any change is readily perceived especially when it begins to evacuate follicular fluid—imagine the large cavity it leaves when it releases its content and the membrane collapses into the cavity. Also note the clear presence of more than two primary proactive follicular blisters in the ovary on the left, which could easily be perceived by palpation.

Finally, we have those heifers in which a single ovary will operate throughout their entire lives. This type of animal is very easy to detect because the active ovary in this phase of sexual development is at least triple the size of the inactive one and also because when it is ovulating the active ovary usually has more than one follicle which may confuse an inexpert hand. But the ovary shrinks in size in the same proportion as the follicle that ovulates shrinks indicating that the animal can be inseminated.

FIG. 35.—Shows a typical example of a heifer of the ovarian genotype characterized by its having a single active ovary throughout its life. Knowing and taking note of this will facilitate the inseminators work in the future, as he will know in advance which horn or ovary is the only one to examine. Observe the large difference in size and how different the ovaries' visible surfaces are. The sterile one is completely smooth, lacks primary follicles, and clearly reveals a hardened appearance.

FIG. 36.—Shows the strange, semi-flat shape of the active ovary, and especially at how the active follicle has developed on the surface; this type of follicle does not display the typical ovulation cavity which usually forms in most cases of ovulation. Nevertheless, it is easier to perceive any change in the tension of the membrane and the size of the follicle. Note also that the left ovary, besides being three times smaller than the active one, does not have proactive follicles, and upon palpation is no more than a hardened mass, indicating its sterility. This facilitates, in the future, deciding which ovary to look at throughout the cow's life.

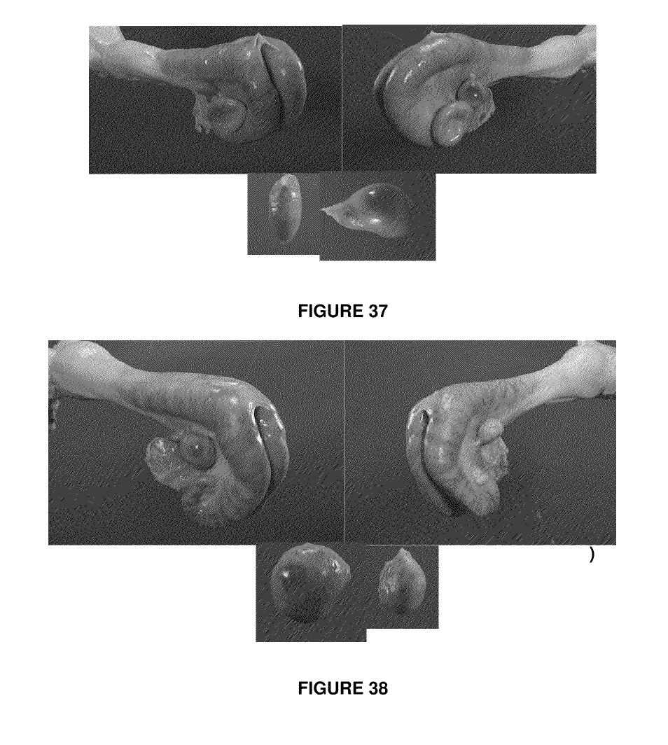

FIG. 37.—This other case shows us the infertility of the ovary on the right by its flattened and smooth structure and total absence of proactive primary follicular blisters, and by its size compared with the other ovary. Notice also that the ovary on the left is the one with the active follicle and that this one, at the time of ovulation, is going to create a radical change in the size of the ovary, which can be used to determine the most propitious moment for insemination.

FIG. 38.—Shows a classic case of an active ovary and a sterile one.

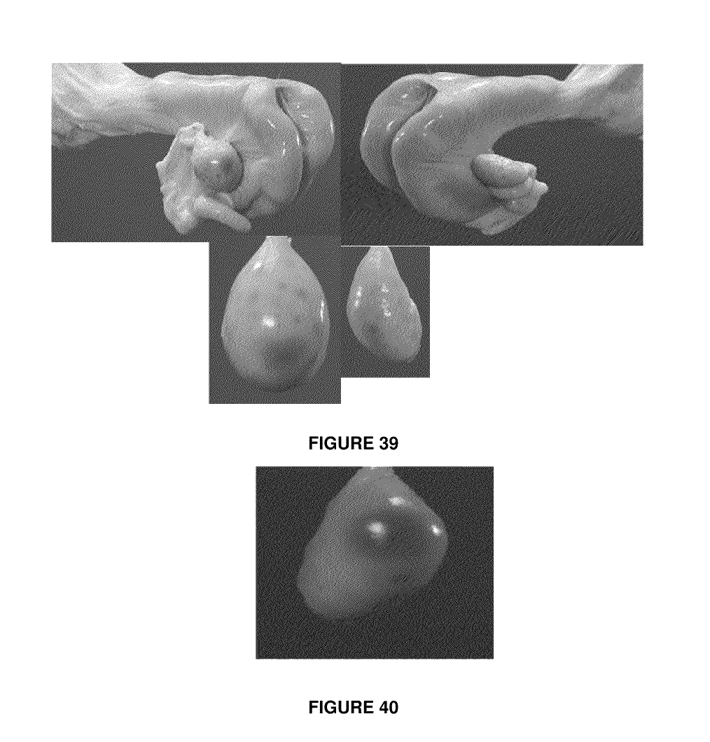

FIG. 39.—Shows an active ovary and a sterile one. Note the presence of two very well developed follicles in the right ovary. In this case the active follicle is very difficult to distinguish because the follicles are almost the same size. It can usually be identified when ovulation has begun.

Heretofore our aim has been to let you know that when palpating a cow that is going out of heat to determine which ovary will ovulate, you will encounter one of the three ovarian genotypes we describe here. This is very important to take note of when a cow is a heifer so that later, in the course of its reproductive life, you will know how it behaves and what you may find when you palpate it again. This is also useful to form an idea of the great variety of sizes and the grotesque shapes that ovaries can assume when the active follicle and the corpus luteum in regression are present in the same ovary, as well as to have an idea of the size that the follicle retains in proportion to the size of the ovary. All of this helps you in one way or another to more easily detect the ovary you are trying to locate

Stages in the Process of Follicular Development after the Heat

In this section, we will describe the changes that take place in an active follicle to determine the optimal time frame and the optimal moment for insemination. We suggest that you get prepared with an insemination glove in order to simulate each one of the stages of follicular development in a small experiment in which you try to perceive the changes that occur in the follicle due to the pressure exerted by the follicular fluid: changes in the size and consistency of the follicle, and changes in the thickness and consistency of the follicle membrane. The idea of this experiment is to play with pressure so that you can feel the differences in the tension of the membrane when it is empty, when it is completely full, and when it is evacuating. This is what happens in real life during each one of the phases into which we have divided the observable events that take place in the follicle after heat.

Phase 1.—Follicular Maturation. This refers to the physical condition that obtains in an active follicle after heat ends, when the follicle reaches its maximum development, which usually occurs in European breeds in approximately nine hours and in zebu breeds in six. The characteristic feature of this stage is that the follicle feels full of follicular fluid as if unable to hold any more liquid. In this phase, the follicle usually goes from being a soft blister that allows the finger to slightly sink into it, to being a tense blister that does due to the high pressure exerted by the follicular liquid on the membrane which itself feels totally tense and thick. Like a swollen blister able to resist any pressure applied to it without bursting. This always happens at the end of heat and usually ends several hours later, although the exact time is different for each cow. FIG. 40 shows the follicle. Note that its center is clearer and more transparent than the rest; this indicates that the membrane is attenuated in this area, which is in the process of thinning. That is why we can see a darker area surrounding it that will become thinner as the follicular fluid exerts more and more pressure on the membrane.

To help understand this event, I have tried to emulate what happens in the follicle during this phase of development using an Al glove; to do this, inflate the glove or, if you prefer, put water into one of its fingers. Then twist the finger to make a tourniquet to form a blister of 3 centimeters at the tip. Hold the blister by the tourniquet and twist it until you feel the plastic membrane become tense. Play with it by applying and then relaxing pressure on it so that you can tell with your forefinger how the membrane feels when it's not full and how it feels when it can't stand any more pressure.

Phase 2: Pre-ovulation. This refers to the physical state that obtains within the follicle when it is ready to evacuate the follicular liquid and the ovule. The characteristic feature of this stage is that the membrane that composes the follicle gets thinner and feels like a flaccid, fragile blister which indicates that in the next few hours the follicle will evacuate all of its contents. This is the fragile phase of the membrane. So take precautions when palpating the ovary at this time, knowing beforehand that the follicle could be in this weakened state. Remember how you should manipulate the ovary before trying to touch the follicle.

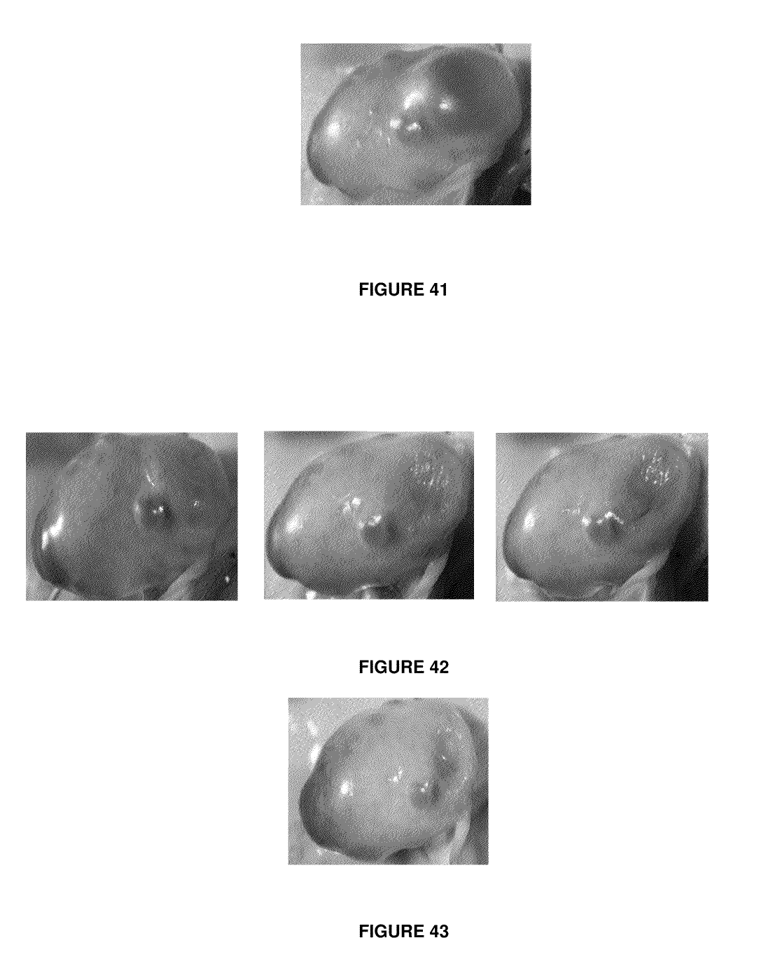

FIG. 41.—Note in this follicle how the entire membrane is translucent and how fragile it seems to be.

Finally, in order that you may imagine and learn to perceive what happens within the living follicle squeeze the plastic blister, until pressure causes the membrane to become thin near the center, showing a small eruption on the surface of the thinnest wall. Then, with the forefinger, try to feel the thinness and the elasticity that characterizes the membrane at this stage which allows a finger to sink into it easily, and then recovers its original shape once pressure is released.

Phase 3: Evacuation. This is the physical condition that obtains within the follicle as the ovulation process unfolds. In this stage, the membrane that forms the outer surface of the follicle loses its tension and collapses, progressively sinking into the follicular cavity as fluid is evacuated. This stage can be detected by palpation due to the cavity the follicle leaves behind when it deflates; it is possible to determine when the stage will end by how much follicular fluid has been evacuated. This event usually lasts four hours after the pre-ovulation phase is detected, and its importance lies in the fact that it defines the time frame and the optimal moment for insemination in the tip of the horn.

FIG. 42.—In this sequence, observe how the follicle membrane is collapsing into the follicle cavity, and how the cavity is closing little by little.

This phase can be simulated by releasing all pressure from the plastic blister you can see and feel that as it deflates the membrane collapses by itself into the cavity.

Phase 4: Fertilization. This is the physical condition that develops in the follicle at the end of the ovulation process. Its characteristic feature is that the membrane rests on the bottom of the follicular cavity without sticking to it. This is easily perceived through palpation by rubbing a finger over the membrane and feeling that it slides smoothly over the surface.

FIG. 43.—Shows how the membrane of the follicle settles on the bottom of the cavity and is beginning to close up. This last phase is simulated with the glove by releasing all pressure and rubbing the bottom of the cavity with the finger to feel how the glove-membrane slides over the bottom.

### Optimal Time Frame for Insemination

This is defined as that period of time or window of opportunity at the technician's disposal during which to inseminate in anticipation of imminent ovulation. This period usually lasts four hours, starting from the moment the follicle is in the pre-ovulation phase and ending just before the membrane settles on the bottom of the ovulation cavity.

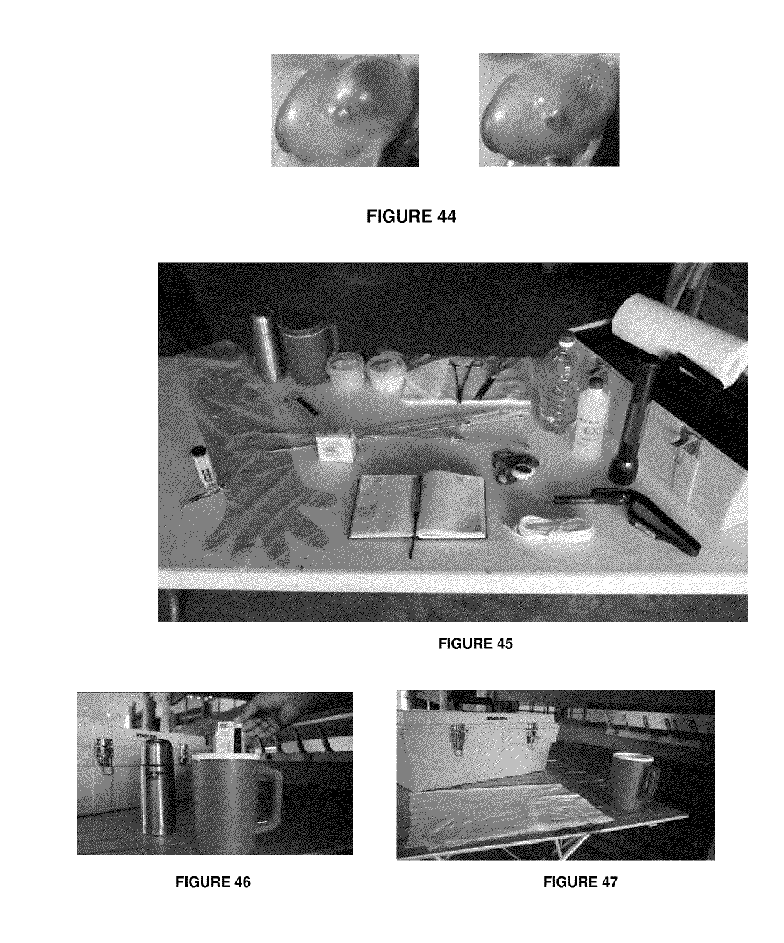

FIG. 44.—Shows the optimal time frame for insemination begins when the follicular blister appears soft and watery. The finger sinks into the blister which quickly resumes its original shape when the pressure is released. It ends when the follicular membrane collapses into the follicular cavity while there is still liquid to be expelled. The importance of this phase is that it is a guide to the handling of the cow in future years, because as soon as you know the beginning and end of this period of time for an individual cow, you will know the schedule by which you must palpate each animal before ovulation, without having to keep checking it all through heat or having to palpate it repeatedly as you have to do the first time you work with an unknown cow.

### Optimal Moment for Artificial Insemination

This moment is defined as that time during which intrauterine conditions allow sperm deposited in the tip of the horn to find a suitable environment in which to strengthen themselves and vigorously navigate unhindered until reaching and fertilizing the ovule. This period lasts approximately one hour and is perceived relatively easily by palpation because the horn is flaccid and floppy and the walls of the horn are very thin, but more importantly because the horn does not respond to rubbing or present any resistance to the friction of the fingers upon being rubbed. This period can also be recognized if, instead of rubbing the horn in one spot, we stroke it all the way up repeatedly. We will see that it responds with an erection that takes a long time to achieve, but quickly disappears if we go back to rubbing it in only one spot. This can be clearly felt as we go from stroking the whole horn to rubbing it in only one spot, because from where the fingers are positioned for rubbing, they will feel how the horn loses its swelling and how the walls get thinner, until the horn turns into a flaccid membrane which permits the fingers to skim the surface freely upon rubbing. Secondly, once you start to analyze the ovary, you will notice that its size has reduced dramatically, that the follicle is empty, and that the follicular membrane covers the follicular cavity without sticking to the bottom and that you can clearly feel it slide freely over the cavity. The optimal moment for insemination, therefore, starts when the follicle has finished evacuating all its liquid and ends when the membrane begins to stick to the bottom of the follicular cavity.

### The Bovine Bioreproductive Clock

The Bovine bioreproductive clock is the genetic memory that defines the reproductive behavior. It is a tool of zootechnical management that studies the physioreproductive constants of the female with the object of precisely predicting those biological events of economic importance for natural and artificial reproduction. It is the precise “clock” with which to measure how long the estrus lasts and to determine the status of the heat and the different stages of the ovulation process. It serves to establish the time frame and the precise moment for natural or artificial insemination.

### Record Log

Animal Identification: name, number or identity code of the animal.
Ovarian Genotype: classification of the ovarian system according to whether it works bilaterally or unilaterally.
AO: Alternating cycles with each new estral cycles
UO-S: Unilateral cycles according to seasons
AO-L: Life-long unilateral cycles
Reproductive Constants The history of the physiological constants of the animal
IBH: The interval between heats expressed in days, the average time of the estral cycle must be measured as exactly as posible.
H: Heat, expressed in hours and corresponds to the average time that the animal accepts copulation.
OVU: Ovulation expressed in hours and refers to the average time that it takes the animal to ovulate after the end of heat.
Optimal Time Frame: Expressed in hours. It is the interval of time between the pre-ovulation and the ovulation phase.
Optimal Moment Expressed in hours, it is the average time in which ovulation occurs after the animal is seen in heat.
Indicator which Facilitate the Inseminator's Work

With the above information, the images, and the simulation practice, the user has already an idea of what happens inside the cow. Now, while you are practicing, all that is left to discuss is a series of indicators that will help you identify the ovary that will ovulate and the best time for insemination.

The first indicator is the fact that at the end of heat, in most cows and heifers, the diameter of the horn that holds the ovulating ovary increases, which makes it easier to locate, especially for an inexperienced inseminator encountering cases where the ovaries are very similar in shape, size and consistency.

The second important indicator tells u show to predict which phase the active follicle is passing through by observing the response of the horn to manual stimulus. In this way, we can determine whether a follicle is still maturing, or is in the evacuation process since all the changes taking place in the follicle are associated with hormonal processes that are manifested in the behavior of the uterus in two different ways.

First, with a violent erection of the uterus that occurs upon touching it. This phase is marked by the fact that local stimulation in any part of the uterus is capable of spreading the erection to the rest of the organ. The intensity of the erection can be sustained merely holding the organ in the hand, all of which indicates that the follicle is in the maturation phase. The uterus in state of repose is flaccid, but clearly demonstrates a tubular configuration coiled upon itself which upon touching is transformed from a flaccid to a tense state manifesting an erection. The tension of the horn's circumference results in a circular horn that prevents its walls from touching each other even the first time it is rubbed. The horn no longer displays any flattened sections but now takes on a conical shape all the way up to the tip. In this state, you can handle the ovary freely, because under these conditions the follicle resists any manual pressure applied to it. Another way manifested is when the erection forms slowly, and only after repeated stroking of the uterus, without the erection ever reaching the intensity that characterized it in the previous phase.

This response, in contrast to the previous one involving rubbing a small spot, is incapable of extending the erection to the rest of the uterus because, though the spot where it is rubbed keeps its tubular shape and poses resistance to the fingertips, it is in the process of posing no resistance. This slow uterine erection characteristically fades away quickly when the uterus is released, and cannot be maintained merely by holding the uterus in your hand. In this process the uterus will gradually lose its tubular shape, because the muscle relaxes to the point of appearing neither tubular nor cylindrical, but rather like a loose membrane that allows the fingers to easily rub each other through its walls. Even though the uterus becomes erect upon stroking it, the erection perceptibly decreases in size and intensity until little by little, as you go from stroking the uterus to rubbing it in one spot. The resistance to the rubbing vanishes, until rubbing becomes effortless and the erection disappears in your hand. All of the above important indicators tell the inexperienced inseminator that the follicle may be in the fragile phase of pre-ovulation. Therefore when palpating the follicle to determine whether it is in the phase of pre-ovulation or in full evacuation and thereby decide when to inseminate the cow, he must exercise the utmost caution to avoid rupturing it.

Another important indicator is the change in size of the ovary during ovulation, which is due to the release of follicular fluid. It is one of the easiest signs to perceive if one is familiar with the ovary at the point of its greatest development and knows how much it shrinks in size as the follicle evacuates.

Finally we should mention, while the inexperienced inseminator is perfecting his techniques through practice, the best way to make an accurate diagnosis is by making use of all of these indicators to correctly ascertain the precise status of the phase the follicle is going through. Likewise, it is important to take into careful consideration the breed of the animals because in our experience, we have observed that it takes pure zebu breeds fewer hours to ovulate than it does pure European breeds. The cows which are a mixture of the two bovine breeds tend to ovulate at intermediate times between these two extremes, in other words, earlier, as in the zebu breeds, in nine hours of fewer, or later, in 16 hours or more, as in the European breeds. This knowledge is of practical use because it helps you experiment with those times at which the cows must be palpated for the first time. While in a few breeds and in some cows you can wait up to 12 hours after their heat ends to do the first palpation, in zebu cattle, you cannot afford to wait more than six hours.

### Steps to Follow Once the Cow Goes Out of Heat:

While you get to know the cows and become experienced in the art of insemination, you must realize it is necessary to palpate the cow at the end of the heat. First, palpate the uterine horns to ascertain whether their reaction is an immediate and violent erection upon touching them, or whether it takes the horns time to react, with an erection that forms slowly and only after repeated manual stimulation. Then check the diameter of the horns, being especially careful to compare the horns when they are perfectly aligned, in order to identify the thicker of the two and to check only the ovary which corresponds to it. If you are just starting to learn, it is better to examine both ovaries, in order to try to identify the ovarian genotype and the consistency of the active follicle membrane so as to learn to determine the next step which might require, which could be the scheduling of another palpation, preparation for insemination or immediate insemination.

### Scheduling Another Palpation:

If upon touching the uterine horns, you feel an immediate and violent erection, an erection which local stimulation causes to extend throughout the reproductive organ and which maintains its intensity—we can safely say that the active follicle is still in development. What this means for the inseminator's operations is that he has at least six hours before ovulation occurs but that he must schedule another palpation at the most suitable time, preferably between the fourth and sixth hours before the ovulation occurs but that he must Schedule another palpation at the most suitable time, preferably between the fourth and sixth hours. Now, if left guided by palpating the ovary with the active follicle, you will find that the membrane which constitutes the follicular blister may or may not be fully tense. Normally, the follicle membrane is flaccid at the end of heat, but from this point on, little by little, it fills with follicular fluid and becomes completely tense, indicating that it is fully mature and that it can be palpated again in another six hours, without fear of missing the optimal moment for insemination.

### Scheduling Insemination:

This technical consideration is mentioned here because it is closely linked to the value of the semen intended for use, especially if it is sexed semen. This applies when you have palpated a follicle in the pre-ovulation stage since it is better to wait until it is already evacuating or better yet until it finishes ovulating to be certain of impregnating the cow or heifer on the first insemination. You will have to do so in accordance with the amount of fluid in the follicle that remains to be evacuated, four hours when it is full, two hours when it is half empty, etc. It is always very important to carry out this middle step of the insemination procedure by checking the follicle, because although the horns may indicate that you are within the optimal time frame, they will not indicate the exact moment when ovulation might occur. If you are palpating a follicle during the pre-ovulation phase when the follicle might be in its most fragile state, it is very important to pay attention to the response of the horns to guide you before moving on to an examination of the ovaries, especially when an erection takes more time to form and does not achieve the same level of intensity than it did before.

### Immediate Insemination:

When the erection of the uterus takes a long time to form upon stimulating it repeatedly, and loses its intensity when you stop stroking the horns, and when the active horn is floppy and does not react to local rubbing with an erection that extends beyond the spot being rubbed, it is in this state that you will perceive that the ovary that has ovulated shrinks dramatically as manifested by how the follicle that was previously full is now a hollow depression into which you can stick your fingertip and feel how easily the membrane slides over the bottom of the cavity. But in these cases, only if the ovary is large enough, as big as or bigger than a peach seed, can these changes be perceived.

### Equipment and Accessories

In this chapter, we will discuss the equipment the materials, and the accessories, necessary to the insemination procedure but we must first consider the number of cows that we intend to inseminate each month, year, or season, or on a schedule by which we synchronize the insemination of cows in herds. This determines not only the number of inseminators we need, but also the amount of semen we must buy, the capacity of our Al tank(s), the number of Al kits we need, and the amounts of other materials to apply needed. We will begin by analyzing the tanks that preserve the frozen semen by considering your necessities and your budget, the number of cows you plan to inseminate, the location of your insemination facility, the location of your farm in relation to the nitrogen supply, and on how long and for what purpose you intend to use the tanks.

First, you have to decide if you need a single tank or more than one to work effectively with the number of cows you have decided to inseminate in the place where you intend to do so. Once you have analyzed all these variables to determine the formula for your particular needs, you will also have to evaluate the technical specifications of the different Al tanks based on their size, weight and nitrogen consumption, in order to choose the most economical in terms of maintenance and transport.

As far as equipment is concerned, one Al kit per inseminator is enough, and the only other thing to consider is whether a portable table on which to place the accessories and tools is required for insemination. Now decide the necessary amount of accessories, such as the French sheaths, the sanitary sleeves, the Al gloves, the lubricant, the paper towels, the alcohol, the crayons, etc. based on the number of cows you are going to inseminate and the quantity of accessories contained in each package.

As far as other supplies go, many have been designed to help detect when a cow is in heat, and we will discuss when we may need them, since this depends less on the number of cows that are inseminated each year, than on what you may lack in terms of personnel—and since many of these supplies have been invented to compensate for staff deficiencies with technology you must get to know and evaluate the different alternatives before buying anything. The most common supplies are markers with paint, which is applied to the rumps of the cows, so that when they are mounted, the paint smears thus showing that the cow is in heat. Likewise there are those accessories that are fastened to the bull's chin (“chin ball”) and leave a mark on the cow in heat every time the bull mounts it. Another way of using paints as heat indicators is painting the animal tail with an oil paint, so that when the cow is mounted, the paint peels off, tearing off hair. Finally, there is the use of crayons to paint the tail so that when the cow is mounted, the mark smudges, and the hair is visibly mussed up. Another type of product to help detect heat is sophisticated electronic equipment, such as heat watch detectors and pedometers, both of which send a signal to a computer that identifies the cows in heat. The former detects heat when the cow is mounted by another animal and the latter when there is an increase in the animal's movements. Nevertheless both have the disadvantage that they can only be used on meek, confined and semi-confined animals. Once we know about the various accessories to help our inseminators with the detection of heat, we have to evaluate whether we need them or not, and whether they are cost-effective for the size and the particular conditions of our operation.

FIG. 45.—Shows the artificial insemination equipment. The kit contains preferably 1. A container, ½ straw semen applicator, ¼ straw semen applicator Al gloves, Alcohol swab container, lubrican swab container, thermometer, thermal glass, thermos for storing hot water, towels, sanitary sleeves, nail clippers, flashlights, headlamp with strap, paper towels, French sheaths, scissors, tongs, stop-watch, lubricant, tail rope, soap, marker, diary, and pen.

Description of the Al Equipment

1. Al box. Container used to hold and protect all the Al equipment from the elements.
2. ½ straw semen applicator. Instrument used to deposit semen contained in a 0.5 cc straw.
3. ¼ straw semen applicator. Instrument used to deposit semen contained in a 0.25 cc straw.
4. Al gloves. Disposable plastic gloves used to protect the hand when inserted into the cow's rectum.
5. Alcohol swab container. Plastic holder used to store cotton swabs soaked in alcohol for cleaning and disinfecting instruments.
6. Lubricant swab container. Plastic holder used to store cotton swabs soaked in lubricant which is used to facilitate the hand's insertion into and its movements inside the cow's rectum.
7. Thermometer. Used to measure the temperature of the water in which the semen is thawed.
8. Thermal glass. Container for heating and maintaining water at the right temperature for thawing the semen.
9. Thermos. Vessel for storing and keeping the water hot.
10. Towel. Cloth on which to spread the insemination equipment out and to protect and keep it clean.
11. Sanitary sleeves. Disposable covers to protect the applicator and keep it from getting soiled with dirt or excrement when it is inserted into the vagina.
12. Nail clipper. To keep nails short to avoid harming mucous membranes of the cow's rectum during insemination.
13. Flashlight. For illumination and detecting cows in heat in the dark.
14. Headlamp with strap. To illuminate the vulva when inserting the semen applicator
15. Paper towels. To dry and protect the applicator from the sun and/or keep it warm in cold weather.
16. French sheathes. Disposable plastic device used to keep the straw inside the applicator when injecting the semen.
17. Scissors. To cut off the tip of the thawed straws.
18. Tongs. To extract the semen straw from the thermal glass.
19. Stopwatch. To measure the thawing time of the straw and to time the preparation of the Al equipment
20. Lubricant. Gel, oil, or soapy water to lubricate the anal sphincter and to allow the hand to penetrate the rectum easily and slides smoothly inside.
21. Tail rope. To tie and fasten the tail of the animal.
22. Soap. To clean the hands.
23. Marker. Dye to mark the haunch on the side where ovulation is occurring.
24. Diary. Log for recording heat and artificial insemination data.
25. Pencil. To take notes with.

### Artificial Insemination

### Step 1:

### Preparation of Semen Defrosting

### Equipment

Equipment: Thermos, thermal container, thermometer. (All these utensils and the following procedure may be substituted if you have an electrical defroster.)

### Procedure

1. Heat water within the container until it is lukewarm, taking care that it reaches a temperature between 35° C. minimum and 37° C. maximum. FIG. 46

### Step 2:

### Preparation of Insemination Equipment

Equipment: Semen applicator (according to the size of the straw to be thawed), scissors, tongs, French sheathes, sanitary sleeves, Al glove, towel, stopwatch, and lubricant.

### Procedure

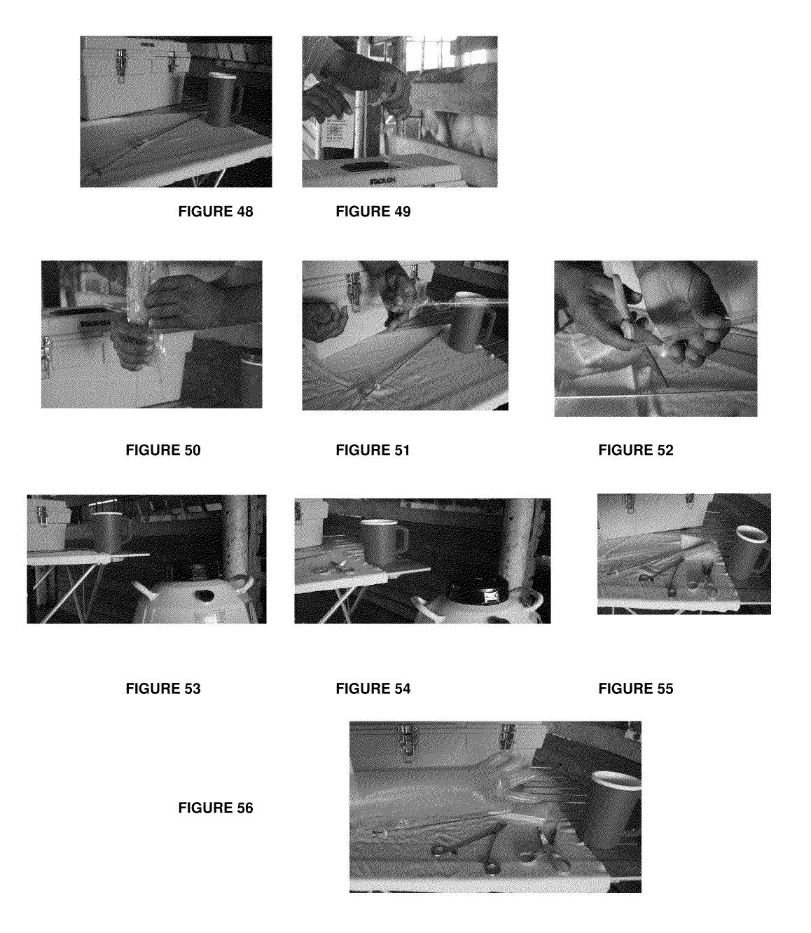

1. Take out and the towel and spread it out on top of the work table. This step is especially important when working in dusty environments. FIG. 47
2. Place the semen applicator on the towel, taking care to pull the piston out, and leave it in the firing position. FIG. 48
3. Take out and detach a sanitary sleeve, being careful not to open it until you insert the French sheath, to keep it from getting dirty. FIG. 49
4. Take a French sheath out of its packaging, being careful not to touch the trailing end of it when you do so and immediately insert it into the sanitary sleeve. Next, cut off 0.5 cm of the trailing end of the sheath, reinsert it into the sanitary sleeve and place it on the towel next to the applicator. Images 50-52.
5. Place the thermal container as close as possible to the Al tank. FIG. 53
6. Place the open scissors to one side of the thermal container. FIG. 54
7. Place the tongs next to the scissors. FIG. 55.
8. Take out a paper towel, fold it into four panels, and place it on top of the cloth towel under the applicator.
9. Take out an Al glove. Inflate it until it is full and leave it ready to use on the table, or if you prefer, in the pocket or the waist of your trousers. FIG. 56
10. Check that all the instruments are laid out in an organized way, so that you can quickly get to any of them when you need them, and so that when you defrost the semen, you won't waste any time trying to find them or have to move around to get them. If it is cold, warm the applicator with your body or by rubbing it briskly with a paper towel before inserting the defrosted straw.

### Step 3: Preparation of the Cow for Insemination

### Equipment: Cattle Chute

### Procedure

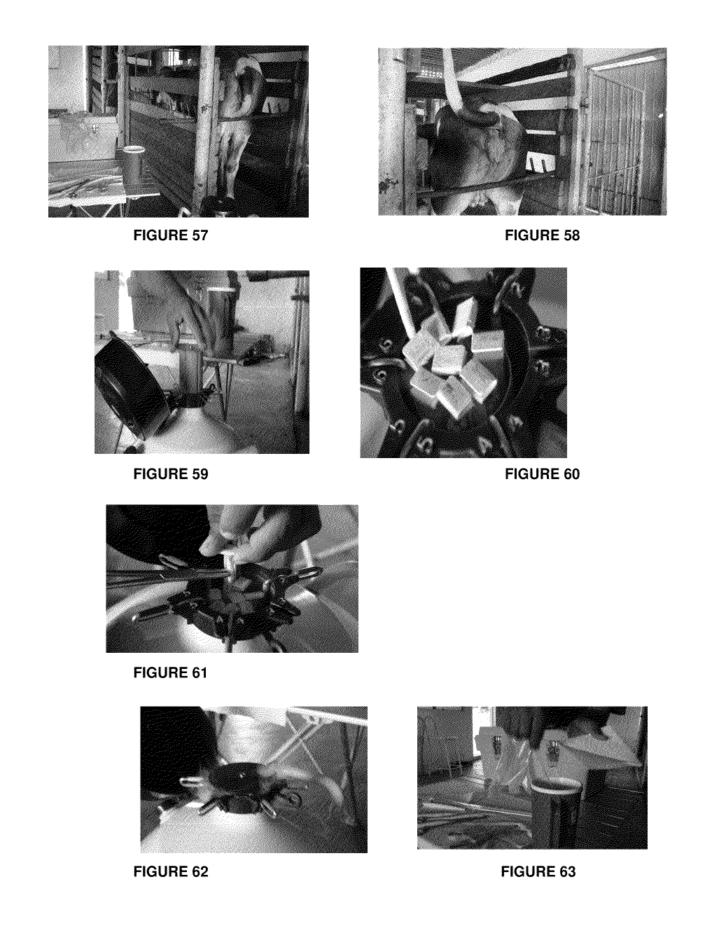

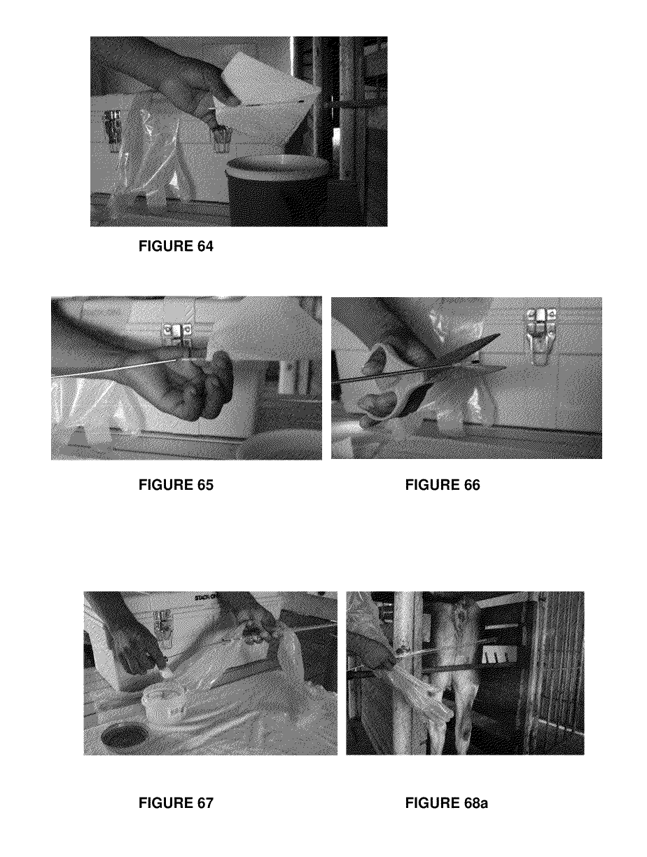

1. Get the cow into the Al cattle chute. FIG. 57
2. Bolt or otherwise restrain the cow in the chute so it cannot move, taking special care that the cross-bar that holds the cow from behind tightens just above the hocks, but far enough away from the vulva to avoid injuring your arm, throw or break the semen applicator.
3. If you have water, wash and dry the cow's hindquarters.
4. Fasten the tail so that it does not obstruct insemination. FIG. 58

### Step 4: Preparation of Semen for Artificial Insemination

Equipment: Al thermos, inventory of semen, stopwatch. All the utensils and procedures are as indicated in step 1.

### Procedure

1. Review the inventory and location of the semen in the Al thermos so that you know in which canister the semen cane you intend to use is located.

### Tank Inventory

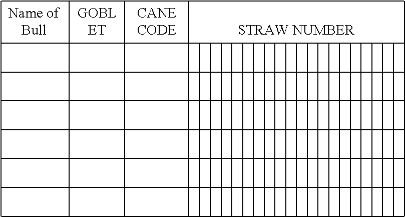

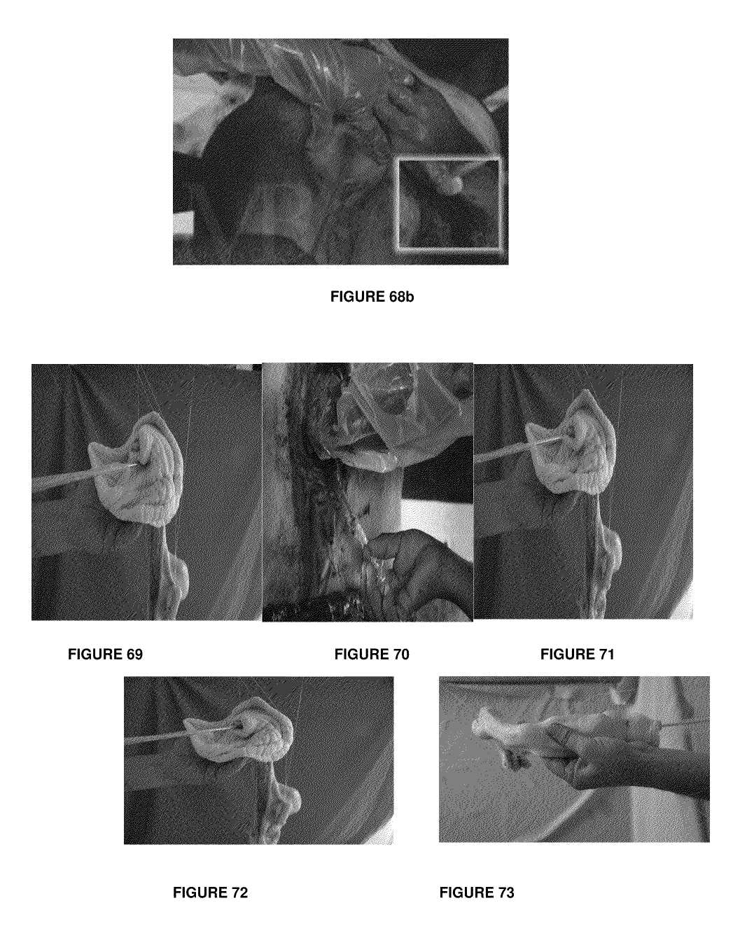

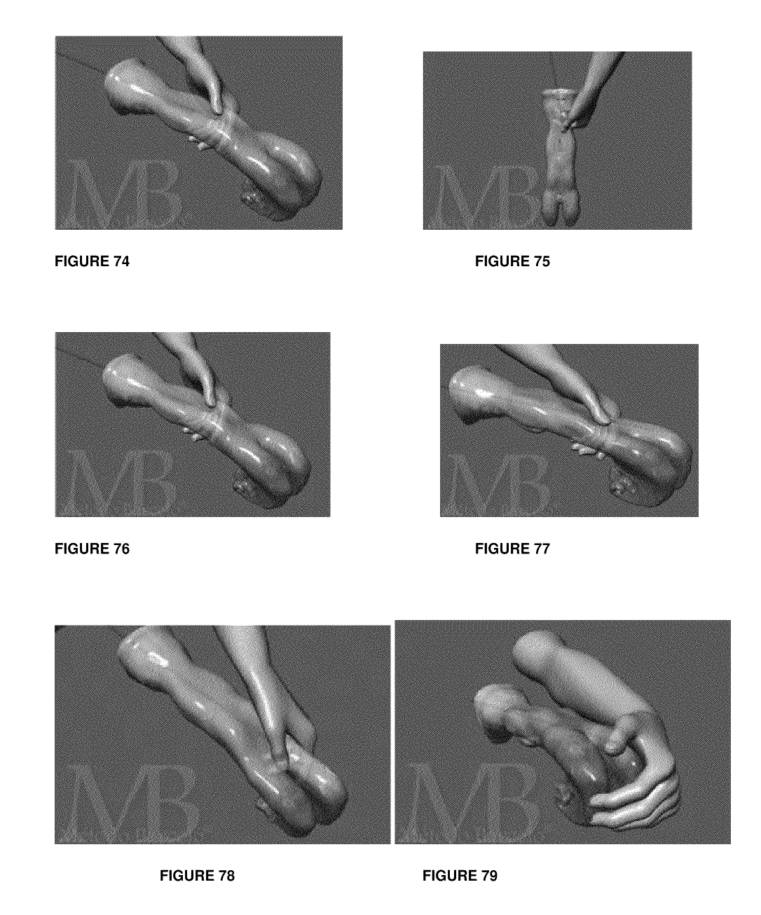

2. Take the top off of the thermos. Lift the canister out without exposing the semen beyond the thermos neck, enough to be able to identify the cane where the semen to be used is located. FIG. 59
3. Take the tongs with the dominant hand, and lift the canister out, holding it as shown to get a good grip on the cane with the tongs. Extract the semen straw and thaw it for 20 seconds in the previously prepared water. As you acquire experience and expertise try not to take more than 45 seconds from between submerging the straw and beginning to insert it into the cow for insemination. Time yourself with a stopwatch that you start as soon as the straw enters the warm water. FIG. 60
4. While the straw thaws, put the canister back in the Al thermos and put the top of the thermos back on. Next, take the paper towel and place it in the palm of your hand, being careful to hold it as shown. Images 61 y 62
5. After waiting 20 seconds to stabilize the temperature of the semen, take the straw with your fingers, remove it from the water and put it on the paper towel to dry it. Then close your hand, folding over the edge of the paper towel to expose the trailing end of the straw, which will be inserted into the applicator. Images 63 y 64
6. Insert the straw into the applicator and with the scissors immediately cut off the tip of the straw about 2 or 3 mm beyond the end of the applicator Images 65 y 66
7. Take the French sheath, making sure that you place your fingers on the retainer of the sheath and not on the sanitary sleeve to keep from perforating it. Then slip it on the applicator until it is fastened tightly by the oppressor ring. FIG. 67
8. With the applicator ready in the dominant hand, take a cotton swab dipped in lubricant into the fingers, being careful not to disturb the cow so that it does not move. Head towards the cow putting on the Al glove. FIG. 68a
9. Open the vulva as shown, taking special care to open it wide enough for the applicator to go in without dragging dirt in with it when it's inserted. Slide the applicator in until at least that part that contains the straw is covered. Immediately squeeze the oil from the cotton swab onto the cow's anus to facilitate inserting the gloved hand into the rectum. Locate and grasp the cervix to stretch the vagina, so that the applicator can slip through to the end. FIG. 68b.
10. Once the applicator is at the bottom of the vaginal sack, pull the sanitary sleeve that covers the applicator until you can tell that the applicator has perforated it and then move the applicator up guided preferably by the thumb to the entry of the cervix, that part known as the radiated flower. Images 69-71.
11. Once the applicator is at the cervix entrance, and the thumb, index, and middle fingers are positioned in front of the entry to the radiated flower, proceed to insert the applicator by gently pushing it until the tip reaches the place where the thumb is positioned. Then move forward again another stretch, pushing the applicator just behind the thumb, index, and middle fingers. Move forward, lifting and adjusting the internal cervical duct, so that the applicator, guided by the thumb, passes through it without causing damage, and without the tip of the French sheath doubling up when you are trying to pass it through irregular ducts. Images 72 and 73
12. When you manage to pass the cervical duct, it is necessary to insert the applicator inside the active horn by first making a small maneuver that involves placing the tip of the applicator in the mouth of the cervix, and moving the back part of the applicator so that the tip points toward the chosen horn. Images 74 and 75
13. Once the applicator is pointing in the right direction, gently push it into the horn, being especially careful to lift and adjust the horn with your index and middle fingers as you go along, pushing the applicator along with the thumb until it is aligned with the index and middle fingers. The applicator should move along just behind the tip of the thumb until it reaches the beginning of the active horn's largest curvature. Images 76 and 77.
14. Once the applicator is just where the horn curvature starts, proceed to raise the horn into position for penetration, with the tip of the horn up, inverted and pointing toward the inseminator. This manipulation process requires practice, to master the skill, but you can do it following a certain prescribed movement of the hand and fingers which we will describe in successive virtual illustrations, showing how to slide the left hand over each one of the horns to reach the tip each horn separately because they are handled differently. We will also show how to grab the tip of the horn with the fingers to raise it and how to turn the hands so the horns will be suitably positioned. Images 78-81

The only requirement is to concentrate on what the hand does inside the rectum to successfully deposit the semen in the tip of the horn without causing damage.

This first sequence illustrates how you slide the left hand along the natural curvature of the left horn, until you get as near as possible to the tip. FIG. 78 shows the hand positioned at the bifurcation from which to start this series of movements; note the position of the hand, thumb, and the silhouette of the position of the other fingers.

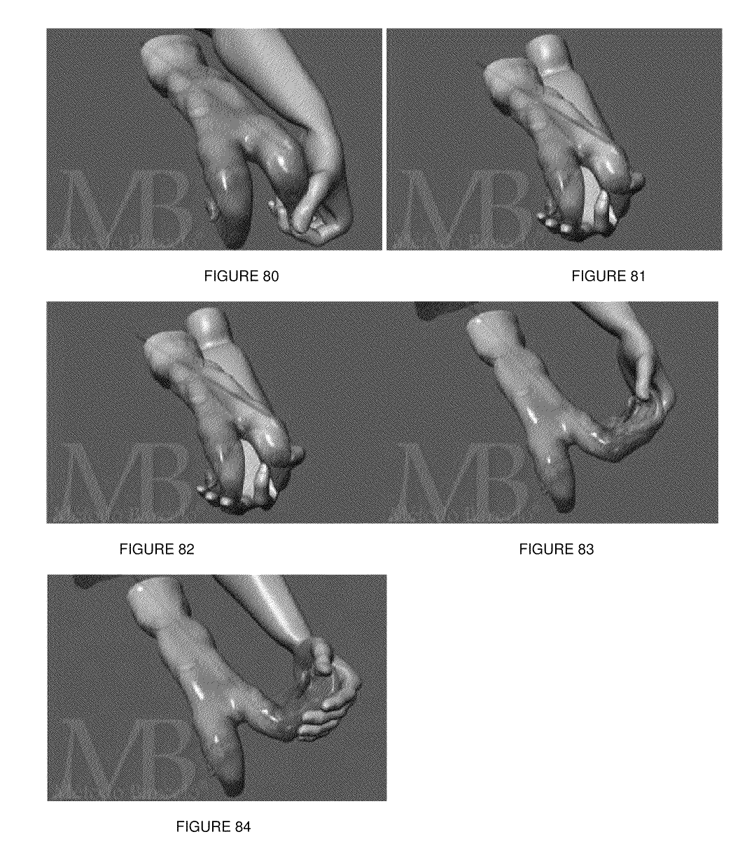

FIG. 79 shows the hand positioned on both horns, this position is achieved with a 180° clockwise rotation of the hand, a movement that begins from the hand position in FIG. 78. FIG. 80 note how the hand is positioned on the left horn, which is achieved by sliding the hand along the natural curvature of the horns until you feel the clear separation of the horns.

Finally, FIG. 81, shows the hand positioned around the tip of the left horn, notice how far the hand must go before you can grasp the tip to raise it.

Images 82-84.—This second sequence illustrates how to grasp and hold the tip of the left horn with your fingers before trying to lift it, where to pull the horn to raise it, and how to rotate the hand to get the horn with the tip facing upwards. FIG. 82 shows how to grab and hold the tip of the left horn firmly: note the position of the thumb and forefinger. FIG. 83 shows the tip of the horn positioned above the level of the rest of the uterus; notice how the uterus appears below the hand, this position is achieved by pulling and lifting the horn in the direction of the inseminator's heart beginning from the position shown in FIG. 8 until you feel you have it up above the rest of the uterus.

Finally FIG. 84 shows the hand positioned with the tip of the horn pointing upwards. This position is obtained with a 180° clockwise rotation of the hand starting from the position illustrated in the previous step

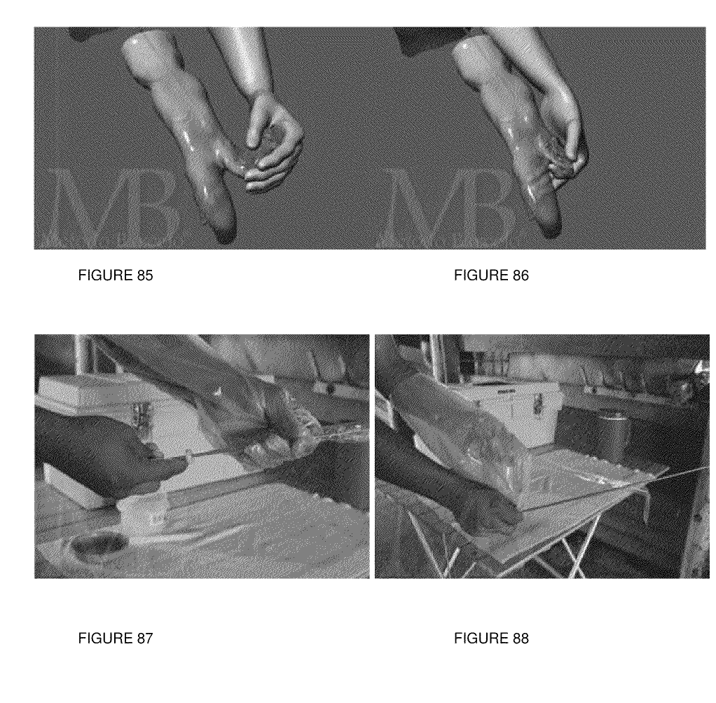

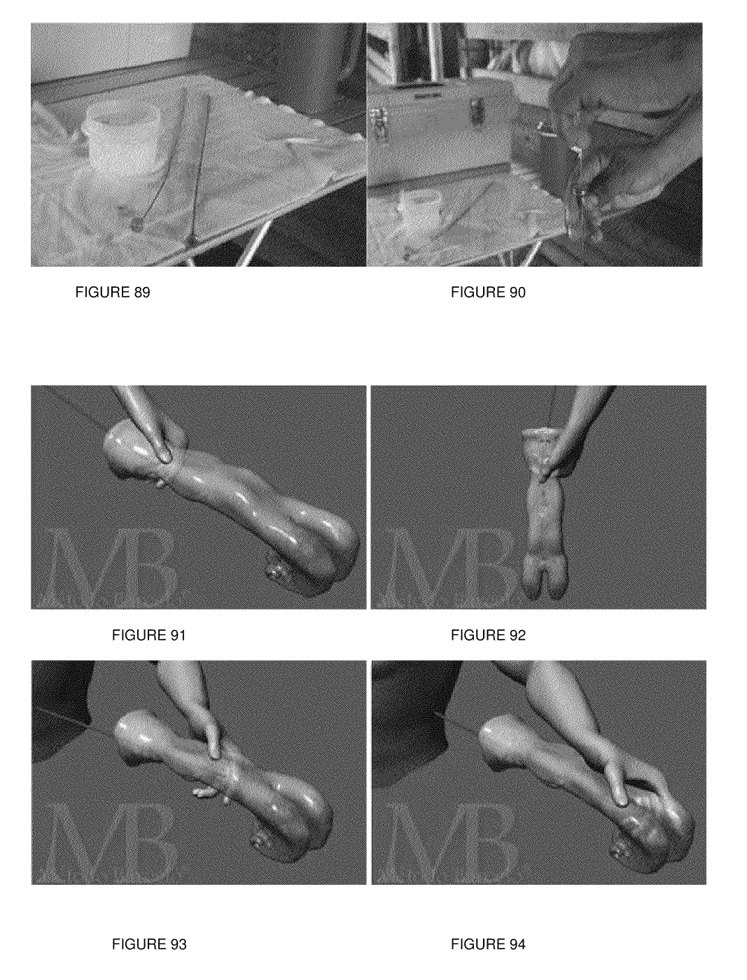

15. Once the tip of the left horn has been positioned upwards, the next step is to place the applicator in the tip of the horn as close as possible to the oviduct to deposit the semen. This step we will explain with a sequence of virtual illustrations of how all the hand and finger movements are executed in order to insert the horn into the applicator. FIG. 85

This sequence shows how to grasp the horn to slip it over the last third of the applicator as close as possible to the entrance of the oviducts. In this step the applicator usually remains stationary while the horn is slipped over it with the thumb and forefinger gripping it firmly on top and slipping it down as far as possible until the fingers are in the position for firing the semen as shown in the last illustration. Note how the thumb and forefinger grasp the tip of the horn so that the semen can be released. Make room for this by stretching the horn or drawing the applicator back, but however you do it, be sure not to block the tip of the applicator; otherwise, the semen will run out inside the French sheath that covers the applicator. FIG. 86.

Finally, as soon as you fire the semen, quickly remove the applicator and dismantle it by loosening the oppressor ring to free the French sheath. Remove the glove and wrap up the sheath with it and throw them in a garbage can. Then wipe the equipment clean as shown below and put it away. Record the data regarding the bull and the insemination in your log and fill out the notation table. Images 87-90

Dismantle the applicator by loosening the oppressor ring to free the French sheathe. Take off the glove, wrap it up with sheathes, and throw them in a garbage can. Then, once you have recorded the data on the bull and the insemination in the table of the log book, clean the equipment as show below, and put it away.

Up until now, we have seen everything we have to do to inseminate the left horn, manipulating it with the left hand. Now it is time to describe how to inseminate at the tip of the right horn, manipulating it with the left hand. We will start from where the hand passes the cervix and moves the applicator to point toward the right horn. Each step and movement will be represented in successive illustrations of two or three images that attempt to demonstrate the positioning of the hand and the fingers at the beginning and at the end of the execution of the movement in question.

This first sequence illustrates the position of the hand as well as the thumb as it guides the applicator to effect penetration of the right horn. Notice in the first illustration the position of the applicator directing the tip toward the right horn, as well as the silhouette of the position of the forefinger, middle finger, and ring finger, which are holding the uterus as the thumb and the applicator move along at the same speed until they are aligned with the forefinger, as illustrated in the second image, just at that point where it begins its passage through the inside of the horn. Images 91-92

This second sequence attempts to illustrate the movements of the fingers and the applicator until the applicator tip can be positioned at the beginning of the right horn's greater curvature. Note in the first illustration how the index, middle, and ring finger in silhouette, are in position to move forward since they lead and open the way for the applicator by lifting and arranging the horn before pushing the applicator forward together with the thumb at the same speed, until they are aligned with the forefinger. Remember that the applicator's penetrating movement must only be made once the lower fingers have straightened the horn out. This movement must be repeated as many times as necessary until reaching the beginning of the curvature. It is a movement that, once you have acquired the manual dexterity, can be performed in a single action by wrapping all your fingers around the horn and sliding the hand and the applicator along until reaching the greater curvature Images 93-94.

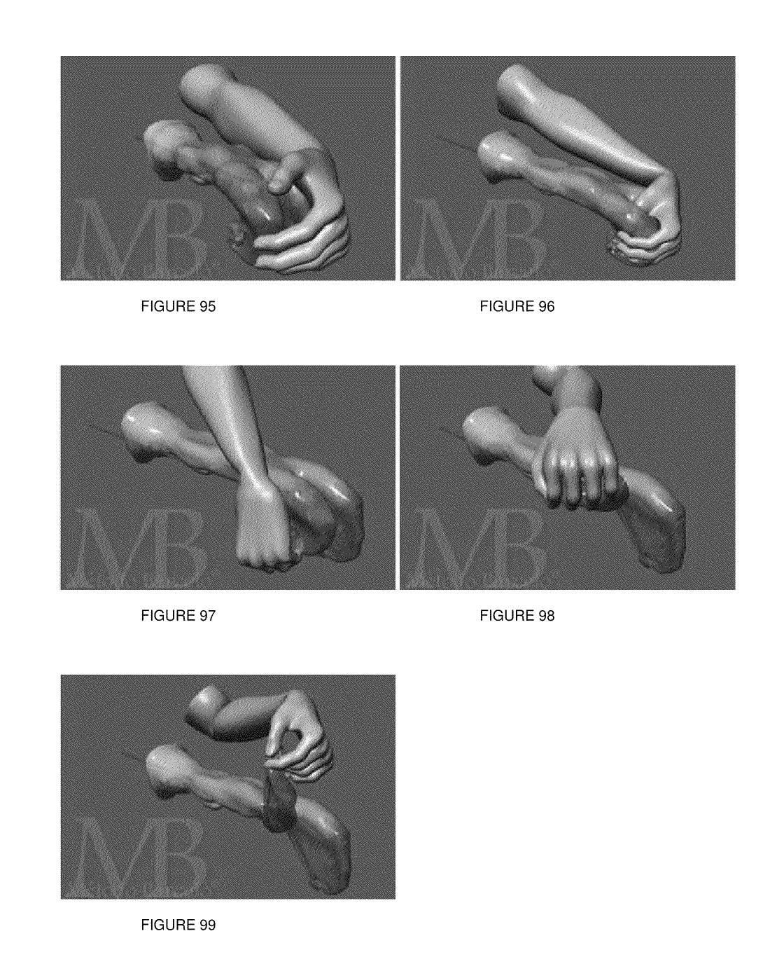

This third sequence of images illustrates the hand movements executed to raise the tip of the right horn. The sequence starts where the hand and the tip of the applicator are in position at the beginning of the bifurcation of the horns. From there the hand rotates clockwise to assume position on top of both horns, covering the upper contours of the horn as shown in FIG. 95. Notice that the hand is positioned in such a way that it can slide along the horn following their natural curvature up to that point where the separation of the horns can be clearly felt. This is the place where you clasp the right horn as shown in FIG. 96, so that the hand can slide as low as possible on the side of the horns, then with another rotation in a clockwise direction with fingers bent in the shape of a hook you catch the right horn as shown in FIG. 97, until it can be grasped firmly between the thumb and forefinger. It is from this position that you can pull and lift the horn in the direction of your heart as shown in FIG. 98, until the horn is positioned as high as possible as in FIG. 99. Leave the horn with the tip facing upward so that with another rotating movement of little more than 90° in a counter-clockwise direction the horn is in position for insemination.

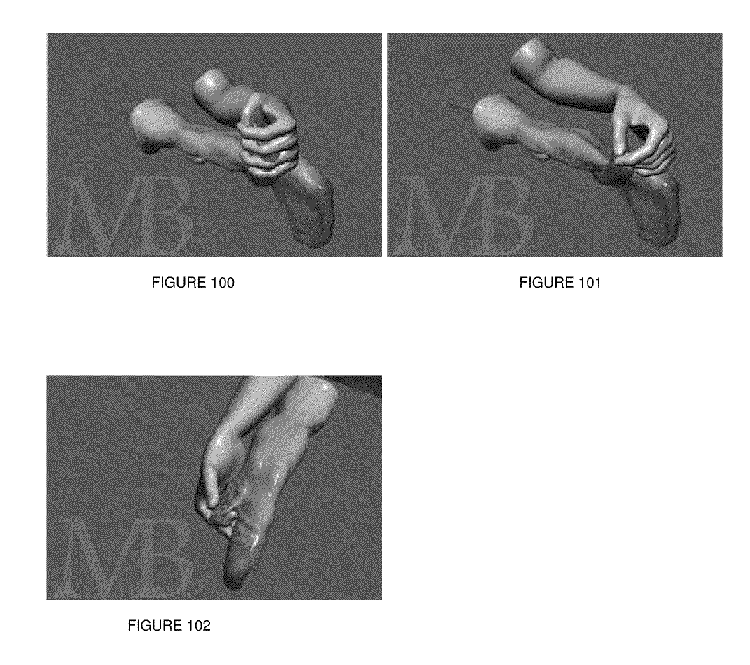

Finally, this last sequence (FIG. 100-102) shows the hand and finger movements which stuff the last third of the horn over the applicator so that the tip of the applicator ends up as close as possible to the oviduct in the firing position. This movement begins by pushing the tip of the horn downward slipping it over the applicator, as indicated in FIG. 100. This movement of going up the horn and then bringing it down over the inseminator is repeated as many times as necessary until the fingers position themselves ahead of the tip of the horn to slip this last part of the horn down over the inseminator and to deposit the semen as close as possible to the oviduct. Finally, it should be noted that every time you pull the horn down over the inseminator to stuff it in you should be careful to straighten out the horn as much as possible so that the inseminator passes through the horn easily until it is in the firing position being careful also to clear the area where the semen will be injected.

### Foundation of the Method for the Invention.

It is based on a series of palpations of the uterine horns, especially the active horns, in order to correlate their status with the physical state of the active follicle until it is determined when to deposit the semen. It begins with the inspection of the horns at the end of the heat to locate in which ovary you find the ovulating follicle. It ends, when we perceive in the active horn, that the follicle has evacuated most of the follicular liquid. In these palpations, we detect first which is the active horn. Then the response of the uterus to the predetermined stimulus to interpret in which phase is the active follicle from the end of the heat to the end of the ovulation process: The phases are:

1st.—Follicular Maturation. It refers to the physical condition of the active follicle at the end of the heat, when it reaches its maximum volumetric development and size. It is characterized because the response of the uterus to predetermined stimulation, is shown as a violent erection as soon as it is touched, both horns form a perfect tubular form of conical aspect to the tip, and the tense of the contour, does not allow the friction between the walls nor the first time it is rubbed. It also has the quality that the local stimulation in any part of the uterus, has the ability to extend the erection to the rest of the body, and the ability to maintain its intensity by the mere fact of holding it on the hand, It is a uterus that at rest is flaccid but clearly shows its tubular configuration coiled upon itself, at this point, it is possible to predict the pressure exerted by the follicular liquid in the membrane that forms the outer surface of the follicle. It feels like a tense blister that does not allow the finger to sink into it, it feels like a swollen blister with thickened wall.

2nd.—Pre-ovulation phase: This refers to the physical condition that happens within the follicle when it is ready to evacuate, it happens normally after the heat. Every female has its own physiological rhythm or schedule for this phenomenon. The characteristic of this stage is that after repeatedly rubbing the uterus, the erection happens slowly without showing the intensity of the previous phase. This phase is marked by the fact that local stimulation is not capable of spreading the erection to the rest of the uterus, though the horns keep its tubular shape and poses resistance to the fingertips. This slow uterine erection characteristically fades away quickly when the uterus is released, and cannot be maintained merely by holding the uterus in the hand. This condition of the uterus is particularly important, because the follicle is located in the phase that is very dangerous to touch it, it can easily break when exploring it. At this point, you can predict that the active follicle is full of follicular liquid. The membrane that composes the follicle gets thinner and elastic, allowing the fingertip to sink into it and resuming its original shape once pressure is released. It is perceived like a flaccid fluid-filled blister.

3rd.—Evacuation phase: It refers to the physical condition that obtains within the follicle as the ovulation process unfolds. In this stage, the uterus starts a process during which progressively loses its tubular configuration to the extent that the follicle begins the process of evacuation of the follicular liquid. The muscle that makes up the horns, relaxes to the extent of not appearing as tubular or cylindrical but a liquid membrane that allows the direct touch of the fingers, and when rubbed it erects again, giving the uterus a tubular shape, though clearly showing that it lost size and intensity. At this point passing from locally touching to rubbing it shows clearly that little by little the resistant to the touch of the fingers fades until it is done without any effort, the erection vanishes on hand even when rubbing. In this phase we can predict, that the membrane that forms the outer surface of the follicle is sinking into the follicular cavity. This event usually lasts four hours after the pre-ovulation phase is detected. This stage can be detected by palpation due to the cavity the follicle leaves behind when touched by the fingertips, enabling us to realize how much follicular fluid has been evacuated. The degree of progress or development of the evacuation process is defined by the fact how deep to immerse the finger in the cavity without pressure.

4th.—Fertilization Phase: This is the physical condition that develops in the follicle at the end of the ovulation process. It is a phase of the uterus that differs from the previous one, because the local rubbing loses the ability to extend the erection or configures a tubular shape, but the horn where ovulation occurred, adopts a flattened, completely flaccid configuration. It does not oppose any resistance to the free touch of the fingers. In this phase we can predict the follicular liquid has been totally evacuated and the membrane that the membrane that forms the visible surface of the follicle rests on the bottom of the follicular cavity without sticking to it. This is easily perceived through palpation by rubbing a finger over the membrane and feeling that it slides smoothly over the surface.

The sequential physical events that are perceived in the uterus to define the moment when we must deposit the sperm take especial importance. Thus the first change that occurs is when the erection changes from violent to slow. Another feature is that at the time to start rubbing partially, the fingertips can brush while it locally configured the tubular form. This local rubbing loses the ability to extend the erection to the rest of the organ. On the other hand if both horns are rubbed, they take a tubular shape opposing some resistance to the fingertips and cannot be maintained merely by holding it in your hands fading quickly when released. At this point, we know that we are in the pre-ovulatory phase, and that the follicle can be easily ruptured when trying to explore it. The next important change, is when the uterus is in a state of repose, it relaxes and loses its tubular and cylindrical configuration and becomes a liquid membrane that allows the direct touch of the fingers, and when rubbed it erects again, giving the uterus a tubular shape. As you go from stroking all over to rubbing in one spot, the resistance to the rubbing vanishes, until rubbing becomes effortless and the erection disappears in your hand.

Finally, we have the event that determines the appropriate time to deposit the semen. It is when the uterus loses its ability to stimulate by stroking all over, it does not capable of erection or configures tubular shape, but the horn where the ovulation took place takes on a flattened and flaccid shape that poses no resistance to the free touch of the fingertips.

The procedure to be patented, It is based on small but significant findings:

1°.—To discover that the physiological process of ovulation can be seen touching the uterus.

2°.—To the discovery of a predetermined stimulation technique of the uterus to determine the phase in which is the process of ovulation.

3°.—The discovery that the exact moment to inseminate is when the active horn adopts a flattened shape that poses no resistance to the free touch of the fingers.

4°.—The discovery that the oviduct is the optimal place for depositing semen.

5°.—The discovery that the gradual changes of the uterus allow us to predict the stages of the follicle, without palpating it.

### Characteristic of the Method:

Given that the method which we intend to patent involves the perception of the events facing the follicle by palpation of the uterus, to decide the time in which the semen must be deposited; This method requires to organize the staff to serve each female according to his own bioreproductive clock; a methodology to follow up the females in heat; a particularly clean equipment, a procedure of preparation of the cow before being inseminated, and; a technique of manipulation of the uterus to achieve successful insemination. Other relevant aspects, though not necessarily essential for the good performance of the method that we intend to patent, are the organization of the herd and the organization of the facilities.

### Organization of the Staff:

Given that this method works with the physiological characteristics of each female and the palpation of the uterus and the stages of the active follicle. The organization of the staff must be without set schedule to evaluate or inseminate the cows. It is strictly necessary that at least one inseminator should be always available during the day and night to inseminate at the right moment. According to the above guidelines, the inseminator (s) could be organized in either of the two following ways: 1st.—Designate the same person to evaluate the uterus at the end of the heat and to follow up the right time for insemination and; 2nd.—Establish a system for the inseminators to record the developments of the uterus of every female. Any system adopted, has to keep record of the number of cows to be inseminated every estrus cycle (25 days); daily and seasonal work load and conditions of the facilities. The organization of the herd, so the staff is in constant and direct contact with the females that will be inseminated for the first time. Locate them in specialized areas that allow easy and constant visual inspection as well as the agile and effective transfer of cows to the insemination site.

### Methodology of the Management of the Females in Heat for Insemination

First Step.—Record the time that the female begins to be mounted.

Second Step.—Record the time in which the female begins to reject mounting.

Third Step.—Palpate the females at the end of the heat to determine in which ovary is located the active follicle. Explore the diameter of the horns in any of the points predetermined by the analysis, and the response of the uterus to the predetermined stimulation, to assess the physical shape and the next step to follow. Following, mark with paint the leg where located the active follicle.

Note: In this first evaluation, at the end of the heat, it is very important to verify the phase of the follicle; some cows could be in ovulating and other in pre-ovulation stage.

As this first palpation decides the next step, and as the criterion of measurement adopted is related to the characteristics of the response of the uterus to the pre-determined stimulus, it is established:

First.—When the uterus responds with a violent erection, that forms as soon as it is touched, it has to be evaluated again at least six hours after the first palpation. This intent is registered in the log as a negative (−).

Second.—When rubbing the uterus, it responds with a slow forming erection, allowing the fingers that are rubbing, to touch without any resistance, while the tubular formation of the uterus prevents it little by little. It has to be evaluated four hours later, register in the log with a plus-minus sign. (+/−).

Third.—When rubbing the uterus, does not present an erection and the active horn is flattened, it is time to inseminate, or set the schedule for insemination. Register in the log with a plus sign 9(+).

Establish a recording system which in practical terms represents what to do with the cow or heifer. The ones marked with a minus (−) sign have to be inspected six hours after the evaluation; the ones marked with the plus-minus (+/−) sign have to be inspected four hours later; the ones with a plus (+) sign have to be immediately inseminated.

Fourth.—This step is subject to the previous assessment of the uterus. If it is logged with a minus (−) sign it requires a new evaluation of the uterus; if it is logged with a (+/−) sign could mean a new evaluation four hours later or ready for insemination.

Fifth.—It always depends on the previous palpation to determine if the cow or heifer is in heat. This last step is the insemination of the cow and it should be logged with a plus (+) sign.

Last Step.—Insemination of cow or heifer.

Therefore, given all the history of the state of the art, and given that the improvements that we intend to claim are a series of innovations that simplify the work of the inseminator, it is no longer important to palpate the follicle; because a technique of manipulation of the uterus allows you to enter the applicator to the oviduct, no matter how long the horns can be. This increases the chances of success at the first intent, because does not run the risk of bursting the follicle when performing the palpation. It reduces the time the inseminator spends following the cow because the new method operates according to the bovine bioreproductive clock. This is a tool that studies the physioreproductive constants of the cow or heifer. It serves to establish the time frame and the precise moment for artificial insemination.

Having sufficiently described my invention, consider it as a novelty and therefore claim of my exclusive property, what it is contained in the following claims:

What it is claimed:

1. A method for artificial insemination of cows and heifers, the method including the following steps:
A) detecting physiological constants of a heat stage and an ovulation phase;
B) detecting which ovary has an active follicle, the determination is done by palpating the cow to:
i) comparing the diameters of each of horn to see which horn is active; or
ii) comparing the sizes of each ovary to determine which ovary is active;
C) manually stimulating an uterus to determine the status of the ovulation phase;
D) accommodating the active horn in position of penetration; and
E) depositing sperms in the oviduct of the uterus indicating the optimal moment to conceive.

2. The method of the claim 1, wherein the optimal moment for artificial insemination is determined by the perception of the physical events of the uterus.

3. The method of the claim 1, wherein the optimal moment for insemination begins when the active follicle evacuates and ends when this process has terminated.

4. The method of the claim 1, wherein each female should be inseminated according to its own bovine reproductive clock.

5. The method of the claim 1, wherein the point of reference to follow up with the females that will be inseminated is the physical state of the uterus after the end of the heat.

6. The method of the claim 1, wherein the palpation of every female in heat to determine the ovary where the active follicle is found, and the consistency, configuration and reaction of the uterine horns to the predetermined stimulus.

7. The method of the claim 1, wherein the stages of the follicle are divided in phases associated with the changes in consistency, configuration and reaction of the uterine horns.

8. The method of the claim 7, wherein a palpation system is established to perceive what happens within the uterus: follicular maturation, pre-ovulation phase, evacuation phase and fertilization phase.

9. The method of the claim 8, wherein the stage of the ovulation phase, the follicular maturation is characterized because the response of the uterus to the pre-determined stimulation is shown as a violent erection as soon as it is touched. Both horns present a perfect tubular shape, of conical aspect to the tip. The tense surface does not allow the friction between the walls the first time that is rubbed. The local stimulation in any part of the uterus extends the erection to the rest of the organ and the ability to maintain its intensity by the mere fact of holding it on the hand. It is a uterus that at rest is flaccid but clearly shows its tubular configuration coiled into itself.

10. The method of the claim 8 wherein the pre ovulation phase refers to the physical state that obtains within the follicle when it is ready to evacuate. The characteristic feature of this stage is that after continuously rubbing the uterus, the erection conforms slowly. The local rubbing loses its ability to extend the erection to the rest of the uterus, but the horns keep its tubular shape against some resistance to the touch of the fingertips. It is also characterized because the erection is not lasting by the mere fact of holding it on the hand and vanishes as soon as we release.

11. The method of the claim 8 wherein in the evacuation phase, refers to the physical condition that obtains within the follicle as the evacuation process unfolds. The uterus begins a process where it loses its tubular configuration, the muscle that forms the horns relaxes to the extent of not appearing tubular or cylindrical, but a liquid membrane that allows the direct touch of the fingers and when rubbed it erects again, giving the uterus a tubular shape.

12. The method of claim 1, further including establishing a registration system of the events, used to predict the schedule of insemination.
